SEQUENCE LISTING (7 sequences)

SEQ ID NO: 1 - protein, 51 aa, Homo sapiens
MKTPWKVLLGLLGAAALVTIITVPVVLLNKGTDDATADSRKTYTLTDYLKN

SEQ ID NO: 2 - protein, 13 aa, Homo sapiens
SRKTYTLTDYLKN

SEQ ID NO: 3 - protein, 12 aa, Homo sapiens
RKTYTLTDYLKN

SEQ ID NO: 4 - DNA, 49 bp, Artificial sequence
  Synthetic oligonucleotide
gtagtcggcccagccggccagtcgcaaaacttacactctaactgattac

SEQ ID NO: 5 - DNA, 47 bp, Artificial sequence
  Synthetic oligonucleotide
gtcggagcggccgcctaaggtaaagagaaacattgttttatgaagtg

SEQ ID NO: 6 - protein, 6 aa, Artificial sequence
  Synthetic peptide
DAAQPR

SEQ ID NO: 7 - protein, 6 aa, Artificial sequence
  Synthetic peptide
DAAQPA

(19) United States
(12) Patent Application Publication
(10) Pub. No.: US 2006/0063719 A1
(43) Pub. Date: Mar. 23, 2006

(54) Methods for treating diabetes

(75) Inventors: Michael I. Jesson, Hopedale, MA (US); Paul A. McLean, Cambridge, MA (US); Glenn T. Miller, Merrimac, MA (US); Barry Jones, Cambridge, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: Point Therapeutics, Inc., Boston, MA (US)

(21) Appl. No.: 11/140,508

(22) Filed: May 27, 2005

Related U.S. Application Data
(60) Provisional application No. 60/622,466, filed on Oct. 27, 2004.
(60) Provisional application No. 60/612,069, filed on Sep. 21, 2004.

(51) Int. Cl. A61K 38/04; A61K 31/69

(52) U.S. Cl. 514/19; 514/64

(57) ABSTRACT

The invention relates to compositions of Glu-boroPro and methods of use thereof in the prevention or management of type 2 diabetes.

### RELATED APPLICATIONS

This application claims priority to U.S. Provisional Applications having Ser. Nos. 60/612,069 and 60/622,466 and filed on Sep. 21, 2004 and Oct. 27, 2004, respectively, the entire contents of both of which are incorporated by reference herein.

### FIELD OF THE INVENTION

The invention relates to the treatment and prevention of conditions that are associated with impaired glucose tolerance, such as type 2 diabetes, using boronic acid compounds.

### BACKGROUND OF THE INVENTION

Type 2 diabetes accounts for 90-95 percent of all diabetes and results from insulin resistance in muscle and impaired function of the pancreatic β-cells that produce insulin in response to dietary sugar (1). In advanced stages of the disease, β-cell function can degenerate to a point where insulin therapy is required.

One potential approach to treatment is to enhance the incretin effect whereby insulin secretion in response to orally ingested glucose is amplified by small peptide hormones. Two gut-derived hormones, glucagon-like peptide-1 (GLP-1) and gastric inhibitory protein (GIP) act through cognate G-protein-coupled receptors on β-cells to potentiate the stimulation of insulin secretion in response to dietary glucose (3).

The incretin effect of both hormones is limited in vivo, however, because they are rapidly inactivated by the serine protease DPP-IV. DPP-IV is a ubiquitously expressed serine protease that can cleave dipeptides from the N-termini of polypeptides in which proline or alanine occupies the penultimate position at the N-terminus (5). A soluble form of DPP-IV is present in blood, and the enzyme is expressed as a 220 kDa type-II integral-membrane protein on the surface of various cell types, including epithelial, endothelial and lymphoid cells (6).

Adequate control of hyperglycemia in patients with type 2 diabetes can attenuate the development of complications such as retinopathy and nephropathy (2). Ideally the goal of treatment should be to intervene when impaired glucose tolerance is initially detected.

### SUMMARY OF THE INVENTION

The invention relates in part to the use of glutamic boroproline (Glu-boroPro) compounds (and compounds related thereto) in the treatment (and prevention) of glucose-associated conditions such as type 2 diabetes. The invention is premised in part on the finding that glutamic acid boroproline compounds are far superior to other compounds including other boroproline compounds in the treatment and prevention of such conditions. This is surprising because of the structural similarity of the compounds tested and their relative equivalence in other assays.

The invention thus provides compositions comprising glutamic acid boroproline compounds (and compounds related thereto) and methods of use thereof for treating and preventing glucose-associated conditions. These conditions include but are not limited to type 1 diabetes (insulin dependent diabetes mellitus or IDDM), type 2 diabetes (non-insulin dependent diabetes mellitus or NIDDM), gestational diabetes, diabetic ketoacidosis (DKA), insulin resistance, impaired glucose tolerance, obesity, hyperglycemia (elevated blood glucose concentration), hyperinsulinemia, hyperlipidemia, hyperlipoproteinemia, and various metabolic syndromes. The invention also intends to embrace treatment of conditions which would benefit from beta cell preservation, reduced glucagon levels or increased insulin availability. These compounds include compounds that when acted upon in vivo release glutamic acid boroproline compounds (e.g., prodrugs of glutamic acid boroproline). Although for convenience and brevity the specification refers to “boroproline” compounds, it is to be understood that the invention intends to embrace compounds containing different functional groups (as described in greater detail herein) such as but not limited to fluoralkylketones, alphaketo amides, alphaketo esters, alphaketo acids, cyanopyrrolidines and thiazolides.

Thus, in one aspect, the invention provides a method for treating a subject having or at risk of developing a glucose-associated condition (such as type 2 diabetes) comprising administering to a subject in need thereof an agent comprising 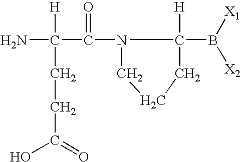
or a prodrug thereof in an effective amount to treat the subject.

In one embodiment, the subject is obese or has impaired oral glucose tolerance. The agent may be administered orally, although other routes of administration are also available. In one embodiment, the agent is administered within 30 minutes of a meal, while in other embodiments, the agent is administered at a time that is independent of food or beverage intake. The agent may be administered at fixed intervals, such as but not limited to every 12 hours, every 24 hours, every 36 hours or every 48 hours.

The agent may be administered in an effective amount that is less than 1 mg/kg/day, less than 500 μg/kg/day, less than 250 μg/kg/day, less than 100 μg/kg/day, less than 50 μg/kg/day, less than 25 μg/kg/day or less than 10 μg/kg/day. It may alternatively be in the range of 1 μg/kg/day to 200 μg/kg/day. In another embodiment, the effective amount is an amount less than the amount required to stimulate cytokine or chemokine induction.

The method may further comprise administering a second agent to the subject. The nature of the second agent will depend on which of the glucose-associated conditions the subject has or is at risk of developing. In one embodiments, the second agent is a second anti-diabetic agent. The agent and the second anti-diabetic agent may be administered in an alternating manner.

In yet another aspect, the invention provides a method for reducing blood glucose comprising orally administering to a subject in need thereof prior to glucose challenge Glu-boroPro having the structure 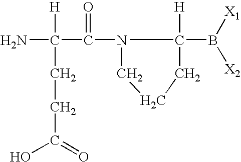
in an effective amount to reduce blood glucose level.

In one embodiment, Glu-boroPro is administered 15 minutes prior to glucose challenge. In one embodiment, the glucose challenge is food or beverage intake. In another embodiment, the blood glucose level is reduced for an extended period of time such as but not limited to 6 hours, 12 hours, 24 hours, 36 hours or 48 hours. In one embodiment, the subject has or is at risk of developing type 2 diabetes. In another embodiment, the subject is obese or has impaired oral glucose tolerance.

In another embodiment, the effective amount is less than 1 mg/kg/day, less than 500 μg/kg/day, less than 250 μg/kg/day, less than 100 μg/kg/day, less than 50 μg/kg/day, less than 25 μg/kg/day or less than 10 μg/kg/day. In yet another embodiment, the effective amount is in the range of 1 μg/kg/day to 200 μg/kg/day. In a related embodiment, the effective amount is an amount that reduces blood glucose at least 40% relative to an untreated subject.

According to another aspect of the invention, a composition is provided that comprises an agent comprising 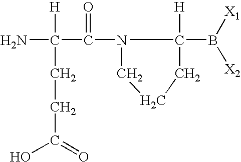
or a prodrug thereof and a second agent, such as but not limited to an anti-diabetic agent.

In one embodiment, the composition further comprises a pharmaceutically-acceptable carrier. In another embodiment, the agent is present in a unit dosage of between 750 μg to 9000 μg. In yet another embodiment, the unit dosage is an amount less than that required to stimulate cytokine or chemokine induction.

In yet another aspect, the invention provides a pharmaceutical composition comprising an agent comprising the structure 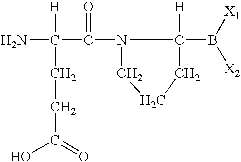
or a prodrug thereof in a pharmaceutically-acceptable carrier and in a unit dosage that is effective for reducing blood glucose.

In one embodiment, the unit dosage is a one a day unit dosage. In a related embodiment, the one a day unit dosage is 750 to 9,000 μg per day. In another embodiment, the unit dosage is an amount that reduces blood glucose by at least 40% as compared to an untreated subject. In another embodiment, the unit dosage is an amount that reduces blood glucose to a level that is +/−10% of blood glucose level in a non-diabetic subject.

In yet another aspect, the invention provides a kit comprising any of the foregoing compositions and agents formulated for oral administration and a daily dispenser. In one embodiment, the composition or agent is formulated as a tablet, pill, capsule or caplet.

In another embodiment, the kit contains a one month supply of the composition. In another embodiment, the daily dispenser is a blister-pack dispenser or a dial dispenser.

Various embodiments apply equally to the different aspects of the invention and these will be recited once for the sake of brevity.

The second anti-diabetic agent may be an insulin, peroxisome proliferator-activated receptor-gamma (PPAR-gamma) agonist, an inhibitor of hepatic glucose production, a stimulator of insulin release from pancreas, a glucosidase inhibitor, or an incretin or incretin analogue.

In some embodiments, the second anti-diabetic agent is an insulin. The insulin may be a rapid-acting insulin, an intermediate-acting insulin or a long-acting insulin. The rapid-acting insulin may be HUMALOG®, HUMALOG® Mix 75/25-Pen, HUMULIN® R, HUMULIN® 50/50, HUMULIN® 70/30, NOVOLIN® R, NOVOLIN® 70/30, NOVOLIN® 70/30 PenFill, NOVOLIN® Innolet, NOVOLOG Mix 70/30, VELOSULIN®, VELOSULIN® BR, ILETIN® I or ILETIN® II. The intermediate-acting insulin may be LENTE® ILETIN® I, LENTE® ILETIN® II, HUMULIN® L, HUMULIN® N, HUMULIN® N pen, NOVOLIN® L, NOVOLIN® N, NOVOLIN®D N PenFill, NPH ILETIN® I, NPH ILETIN® II or NPH-N. The long-acting insulin may be ULTRALENTE®, HUMULIN® U, or Lantus Injection.

In another embodiment, the second anti-diabetic agent is a PPARγ agonist. The PPARγ agonist may be a thiazolidinedione such as but not limited to Avandamet (combination of rosiglitazone and metformin), rosiglitazone (Avandia), pioglitazone (Actos), troglitazone (Rezulin), (S)-((3,4-dihydro-2-(phenyl-methyl)-2H-1-benzopyran-6-yl)methyl-thiazolid-ine-2,4-dione (englitazone), 5-{[4-(3-(5-methyl-2-phenyl-4-oxazolyl)-1-oxo-propyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189),5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,-4-dione (AY-31637), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-y-1)ethoxy)phenylmethyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)ethoxy)phenyl]methyl}-thiazolidine-2,-4-dione (rosiglitazone), 5-{[4-(2-(5-ethyl-2-pyridyl)ethoxy)phenyl]-methyl-}thiazolidine-2,4-dione (pioglitazone), 5-{[4-((3,4-dihydro-6-hydroxy-2,5,-7,8-tetramethyl-2H-1-benzopyran-2-yl)methoxy)-phenyl]-methyl}-thiazolidine-2,4-dione (troglitazone), 5-[6-(2-fluoro-benzyloxy)-naphthalen-2-ylmethyl-]-thiazolidine-2,4-dione (MCC555),5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297). The PPAR-gamma agonist may also be a natural prostaglandin D(2) (PGD(2)) metabolite, 15-deoxy-Delta(12, 14)-prostaglandin J(2) (15d-PGJ(2)).

In another embodiment, the second anti-diabetic agent is an inhibitor of hepatic glucose production. The inhibitor of hepatic glucose production may be a biguanide such as but not limited to metformin (GLUCOPHAGE), Avandamet tablet, Glucovance tablet, or Metaglip tablet.

In yet another embodiment, the second anti-diabetic agent is a stimulator of insulin release from pancreas such as but not limited to a sulfonylurea or a meglitinide. The sulfonylurea may be acetohexamide (DYMELOR), chlorpropamide (DIABINESE), tolbutamide (ORINASE, RASTINON), glipizide (GLUCOTROL, GLUCOTROL XL), glyburide (DIABETA; MICRONASE; GLYNASE), glimepiride (AMARYL), glisoxepid (PRO-DIABAN), glibenclamide (AZUGLUCON), glibomuride (GLUBORID), tolazamide, carbutamide, gliquidone (GLURENORM), glyhexamide, phenbutamide, tolcyclamide or gliclazide (DIAMICRON). The meglitinide may be Repaglinide (PRANDIN) or nateglinide (STARLIX).

In a further embodiment, the second anti-diabetic agent is a glucosidase inhibitor such as but not limited to acarbose (PRECOSE, GLUCOBAY), miglitol (GLYSET, DIASTABOL) or voglibose.

In yet another embodiment, the second anti-diabetic agent is an incretin or incretin analogue. The incretin or incretin analogue may be GLP-1, GIP, EXENATIDE or EXENATIDE LAR.

In still another embodiment, the second anti-diabetic agent is a DPP-IV inhibitor selected from the group consisting of alanyl pyrrolidine, isoleucyl thiazolidine, and O-benzoyl hydroxylamine.

In one embodiment, the agent is 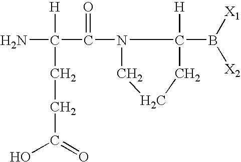
wherein the C bonded to the B is in the R-configuration and preferably the glutamic acid constituent is in the S-configuration.

In another embodiment, the agent is a prodrug of Glu-boroPro. For example, the agent may be a cyclic version of Glu-boroPro, an ester of Glu-boroPro, a boroxine molecule, or an alcohol precursor of Glu-boroPro.

In a related embodiment, the agent has a structure 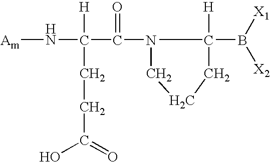
wherein Aₘ is any naturally or non-naturally occurring amino acid bonded in either an S- or an R-configuration or a peptide or peptidomimetic; m is an integer equal to or greater than zero, such that when A is an amino acid residue and m is greater than one, each A in Aₘ may be a different amino acid residue from every other A in Aₘ; and each X₁ and X₂ is, independently, a hydroxyl group or a group capable of being hydrolyzed to a hydroxyl group in aqueous solution at physiological pH. Preferably, the bonds between amino acid residues of A are peptide bonds.

In another related embodiment, the agent has a structure 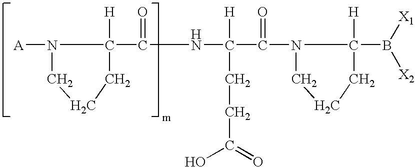
wherein A is any naturally or non-naturally occurring amino acid in an S- or an R-configuration or a peptide or peptidomimetic; m is an integer greater than or equal to zero, provided that when A is an amino acid residue and m is greater than one, A in each repeating bracketed unit can be a different amino acid residue; and each X₁ and X₂ is, independently, a hydroxyl group or a group capable of being hydrolyzed to a hydroxyl group in aqueous solution at physiological pH. Preferably, the bonds between amino acid residues are peptide bonds.

The agent may comprise an S-enantiomer of glutamic acid. In important embodiments, the agent comprises a R-enantiomer of boron substituted pyrrolidine. The agent may further comprise a mixture of R- and S-enantiomers of boron substituted pyrrolidine. In a related embodiment, the mixture of R- and S-enantiomers of boron substituted pyrrolidine contains at least 95%, at least 96%, at least 97%, at least 98%, or at least 99% of the R-enantiomer of boron substituted pyrrolidine.

Subjects to be treated are mammals susceptible to glucose-associated conditions. These include animals, although in most embodiments humans are preferred. Human subjects include adults, juveniles, infants and fetuses.

Thus, in yet another aspect, the invention provides a method for treating a subject having type 2 diabetes comprising orally administering to a subject in need thereof, 15 minutes prior to glucose challenge, an agent having a structure of 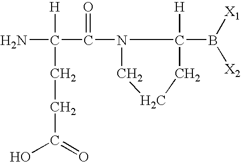
wherein each X₁ and X₂ is a hydroxyl group, in an amount effective to reduce blood glucose level, after glucose challenge, by at least 40% relative to an untreated subject (i.e., an untreated subject having type 2 diabetes).

These and other aspects of the invention, as well as various advantages and utilities, will be more apparent with reference to the detailed description of the invention. Each aspect of the invention can encompass various embodiments, as will be understood.

### BRIEF DESCRIPTION OF THE DRAWINGS

FIG. 1A is a graph showing the level of DPP-IV activity in vitro as a function of concentration of the indicated amino acid boroPro compounds.

FIG. 1B is a graph showing the level of DPP-IV activity in vitro as a function of time after exposure of DPP-IV to the indicated amino acid boroPro compounds.

FIG. 2A is a graph showing the level of DPP-IV activity in vivo as a function of dose of Glu-boroPro.

FIG. 2B is a graph showing the level of DPP-IV activity in vivo as a function of time after exposure to Glu-boroPro.

FIG. 3 is a graph showing the level of G-CSF produced following in vitro exposure of human bone marrow stromal cells to the indicated amino acid boroPro compounds.

FIG. 4A is a graph showing the level of serum DPP-IV activity in vivo at 2 hours after administration of the indicated doses of various amino acid boroPro compounds.

FIG. 4B is a graph showing the level of serum KC in vivo at 2 hours after administration of the indicated doses of various amino acid boroPro compounds.

FIG. 5A is a histogram showing the level of DPP-8 activity in vitro following exposure to Val-boroPro and Glu-boroPro.

FIG. 5B is a graph showing the level of DPP-8 activity in vitro as a function of time after exposure to the indicated amino acid boroPro compounds.

FIG. 6A is a graph showing the level of blood glucose in vivo as a function of time following administration of Glu-boroPro and an oral glucose challenge.

FIG. 6B is a histogram showing the level of area under the curve (AUC) following in vivo exposure to Glu-boroPro.

FIG. 7A is a graph showing the level of DPP-IV activity in vivo as a function of time immediately following administration of Glu-boroPro and an oral glucose challenge.

FIG. 7B is a graph showing the level of DPP-IV activity in vivo as a function of time (longer time interval) following administration of Glu-boroPro and an oral glucose challenge.

FIG. 7C is a graph showing the level of blood glucose in vivo as a function of time following administration of Glu-boroPro and an oral glucose challenge.

FIG. 7D is a graph showing the level of insulin in vivo as a function of time following administration of Glu-boroPro and an oral glucose challenge.

FIG. 7E is a graph showing the level of GLP-1 (1-36) in vivo as a function of time following Glu-boroPro and an oral glucose challenge.

It is to be understood that the drawings are not required for enablement of the invention.

### DETAILED DESCRIPTION OF THE INVENTION

The invention relates to the treatment and prevention of conditions that are associated with abnormal glucose tolerance, absorption, metabolism, utilization and the like. These conditions are referred to as glucose-associated conditions.

Glucose-associated conditions include but are not limited to type 1 diabetes (insulin dependent diabetes mellitus or IDDM), type 2 diabetes (non-insulin dependent diabetes mellitus or NIDDM), gestational diabetes, diabetic complications such as metabolic acidoses (e.g., diabetic ketoacidosis (DKA)), carbohydrate and lipid metabolism abnormalities, glucosuria, micro- and macrovascular disease, polyneuropathy and diabetic retinopathy, diabetic nephropathy, insulin resistance, impaired glucose tolerance (or glucose intolerance), obesity, hyperglycemia (elevated blood glucose concentration), hyperinsulinemia, hyperlipidemia, hyperlipoproteinemia, atherosclerosis and hypertension (high blood pressure) related thereto, and various metabolic syndromes. Metabolic syndromes include digestive tract diseases such as ulceric or inflammatory disease; congenital or acquired digestion and absorption disorder including malabsorption syndrome; disease caused by loss of a mucosal barrier function of the gut; and protein-losing gastroenteropathy. Ulceric diseases include gastric ulcer, duodenal ulcer, small intestinal ulcer, colonic ulcer and rectal ulcer. Inflammatory diseases include esophagitis, gastritis, duodenitis, enteritis, colitis, Crohn's disease, proctitis, gastrointestinal Behcet, radiation enteritis, radiation colitis, radiation proctitis, enteritis and medicamentosa. Malabsorption syndrome includes essential malabsorption syndromes such as disaccharide-decomposing enzyme deficiency, glucose-galactose malabsorption, fructose malabsorption; secondary malabsorption syndrome, short gut syndrome, cul-de-sac syndrome; and indigestible malabsorption syndromes such as syndromes associated with resection of the stomach, e.g., dumping syndrome. Other conditions associated with above-normal blood glucose concentration either in an acute or chronic form are also embraced by the invention. The invention also intends to embrace treatment of conditions which would benefit from beta cell preservation, reduced glucagon levels or increased insulin availability.

Diabetes is generally a disease in which the body is not able to produce or does not adequately utilize insulin. Insulin is a hormone that facilitates entry of sugars, starches and the like into cells, thereby allowing their conversion into useable energy for the body. In diabetes, therefore, there is a buildup of glucose in the blood due to the inefficient or nonexistent cellular uptake of sugar, starches and the like. Type 2 diabetes is also characterized by progressive beta-cell failure. Type 2 diabetes is also referred to as adult onset diabetes or non-insulin-dependent diabetes (NIDDM).

It was found according to the invention that a particular boronic acid containing compound, Glu-boroPro, exhibited a combination of potency and duration of DPP-IV inhibition that was significantly better than that of other known amino boronic dipeptides. This difference in activity between the amino boronic dipeptides tested was surprising because the compounds are structurally similar and behave relatively equivalently in other assays (e.g., DPP-IV inhibition). The potential of Glu-boroPro to treat type 2 diabetes and other glucose-associated conditions was indicated in rodent models in which the compound was shown to control blood glucose levels and stimulate insulin and GLP-1 (1-36) levels following oral glucose challenge. These assays provide surrogate readouts that enable the determination of the anti-diabetic activity of compounds in vivo. Glu-boroPro also demonstrated suitable pharmacological properties and specificity of action, making it even more appropriate for in vivo use in the management of glucose-associated conditions such as type 2 diabetes.

Although not intending to be bound by any particular mechanism or theory, DPP-IV is presumed to be the target of Glu-boroPro compounds. DPP-IV is responsible for the rapid N-terminal degradation of GIP and GLP-1(t_{1/2}˜1 min) in vivo (4). DPP-IV is therefore a molecular target for compounds designed to amplify the biological activity of GLP-1 and GIP (4). Because resistance to the activity of GIP appears to develop in glucose-associated conditions such as type 2 diabetes, it is currently thought that inhibition of DPP-IV will mainly impact the activity of GLP-1. Because GLP-1 is an incretin that stimulates insulin production by pancreatic β-cells in response to the oral intake of glucose (7), DPP-IV plays a physiological role in the regulation of blood glucose levels. This has been validated by the demonstration of no N-terminal degradation of GLP-1 and enhanced insulin secretion in response to oral glucose challenge in DPP-IV-null mice generated by homologous recombination (8). GLP-1 also inhibits glucagon synthesis and gastric emptying, promotes the growth of pancreatic islets and β-cells, and may have an anorexic effect by acting on the hypothalamus. DPP-IV inhibitors may amplify these other biological activities of GLP-1. As a result, the invention embraces methods for inducing weight loss, particularly in obese subjects regardless of whether such subjects are diabetic or not.

Again, although not intending to be bound by any particular theory or mechanism, the invention further embraces the use of modified compounds that do not enter cells but which show enzyme inhibitory capacity similar to that of Glu-boroPro containing compounds. These modified compounds may derive from compounds known to enter the cell and known to have enzyme inhibitory activity (such as for example against DPP-IV). Modification can include changing the overall charge of these compounds or creating compounds that are sterically precluded from cell entry. Other compounds embraced by the invention include those having an overall charge similar to Glu-boroPro at physiological pH, and preferably, structural and size similarity with Glu-boroPro.

The agents of the invention include Glu-boroPro compounds. A Glu-boroPro compound is a compound that contains a glutamic acid bound via a carboxy (C) terminal bond to a pyrrolidine bound to a boronic acid or a boronic ester. For the sake of convenience and brevity, various aspects and embodiments of the invention refer to Glu-boroPro compounds but it is to be understood that other compounds related to Glu-boroPro compounds (e.g., prodrug compounds and alternatively substituted compounds) are also embraced by the invention and can be equivalently used in the aspects and embodiments described.

Glu-boroPro has a structure as follows: 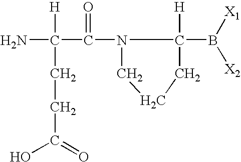
wherein each X₁ and X₂ is, independently, a hydroxyl group or a group capable of being hydrolyzed to a hydroxyl group in aqueous solution at physiological pH. The bond between carbon in the pyrrolidine and the boron can be in an S-configuration, but it is preferably in the R-configuration. The peptide bond between glutamic acid and the pyrrolidine can be in the R-configuration, but in some embodiments it is preferably in the S-configuration. In some embodiments, X₁ and X₂ are hydroxyl groups. Glu-boroPro therefore includes L-Glu-R-boroPro, D-Glu-R-boroPro, L-Glu-S-boroPro and D-Glu-S-boroPro.

Accordingly, the compound can have the following structure showing an S-R configuration (i.e., the glutamic acid to pyrrolidine bond is in the S-configuration and the carbon to boron bond is in the R-configuration):

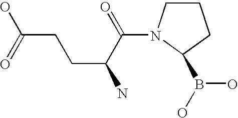

Glu-boroPro can also be provided in cyclic form, which is then converted into a linear form upon in vivo administration, particularly once exposed to an acidic environment such as the stomach. Cyclic amino boronic acids are described in greater detail in U.S. Pat. No. 6,355,614 B1, issued Mar. 12, 2002, the entire contents of which are incorporated by reference herein. The linear and cyclic forms of Glu-boroPro compounds are provided in solution or dry form. Linear and cyclic forms of Glu-boroPro may be in equilibrium. A cyclic Glu-boroPro can have the following structure:

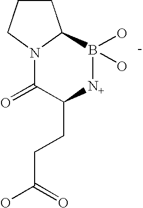

The agents of the invention also embrace Glu-boroPro containing compounds as well. A Glu-boroPro containing compound is an agent that comprises Glu-boroPro (as defined above). One class of Glu-boroPro containing compounds comprises Glu-boroPro bound to additional amino (N) terminal naturally or non-naturally occurring amino acid residues or peptides or peptidomimetics. A general formula for this class of compounds is 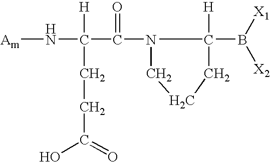
wherein A is any naturally or non-naturally occurring amino acid or peptide or peptidomimetic bonded in either an S- or an R-configuration, m is an integer equal to and preferably greater than zero, such that when m is greater than one and A is an amino acid residue, each A in Aₘ may be a different amino acid residue from every other A in Aₘ; and each X₁ and X₂ is, independently, a hydroxyl group or a group capable of being hydrolyzed to a hydroxyl group in aqueous solution at physiological pH. The C bonded to B can be an S-configuration but preferably it is an R-configuration. In some important embodiments, the peptide bonds between amino acids are in the S-configuration. If such peptide bonds include serine or cysteine, then such bond may be in the R-configuration. In some embodiments, X₁ and X₂ are hydroxyl groups.

In some important embodiments, m is equal or greater than two, or it is a multiple of two (e.g., 2, 4, 6, 8, 10, etc.), or it is a repeating dipeptide having a proline residue at the C terminal (e.g., A-Pro). In some preferred embodiments, the general formula for such compounds is 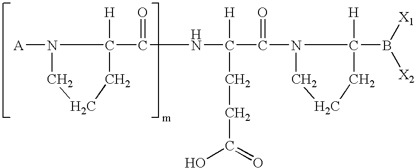
wherein A is any naturally or non-naturally occurring amino acid or peptide or peptidomimetic in an S- or an R-configuration; m is an integer (including zero), provided that A in each repeating bracketed unit can be a different amino acid residue; the bonds between residues are peptide bonds; and each X₁ and X₂ is, independently, a hydroxyl group or a group capable of being hydrolyzed to a hydroxyl group in aqueous solution at physiological pH. In some embodiments, the glutamic acid chiral center is in the S-configuration. Glu-boroPro can also be attached to 3, 5, 7, 9, etc. amino acid residues.

Glu-boroPro compounds (including Glu-boroPro) in some instances may be substantially optically pure. That is, at least 90%, 92%, 94%, 95%, 96%, 97%, 98% or 99% of the carbon atoms bearing boron are of the R-configuration in some embodiments.

A synthesis scheme for making the enantiomers of the invention is as follows:

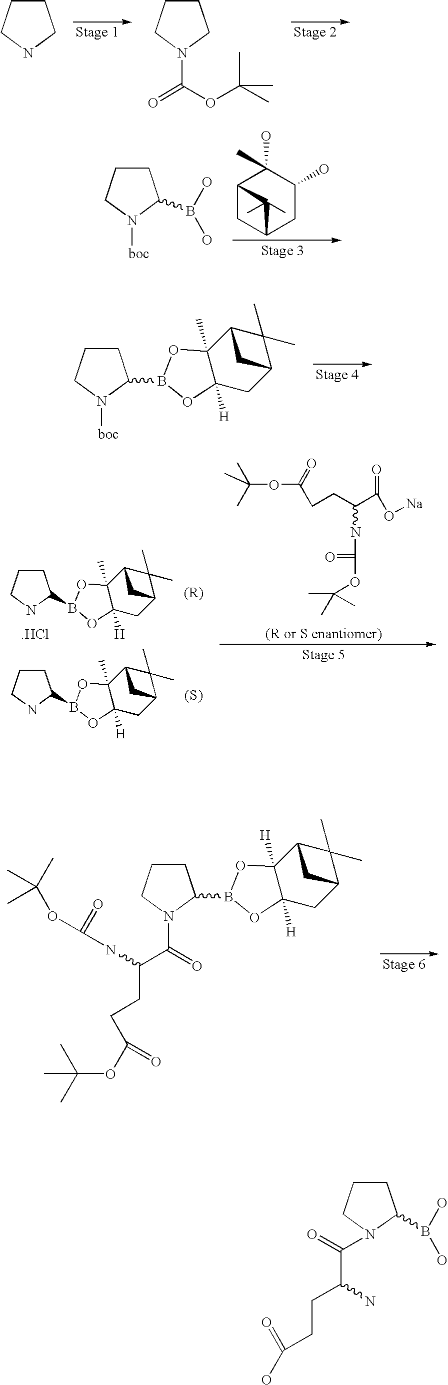

Further methods for synthesizing optically pure isomers of these agents are disclosed in Coutts et al. J. Med. Chem., 1996, 39:2087-2094 and in published PCT application WO93/10127, published May 27, 1993 and in published PCT application WO 93/08259. As will be understood to those of ordinary skill in the art, the compounds of the invention can be synthesized using D- and preferably L-isomers of glutamic acid and proline.

Glu-boroPro containing compounds also embrace prodrugs of Glu-boroPro. A prodrug of Glu-boroPro as used herein is a compound that is metabolized in vivo to Glu-boroPro or disintegrates (e.g., upon contact with stomach acid) to form Glu-boroPro. Some prodrugs are converted into Glu-boroPro via hydrolysis or oxidation in vivo. These include alcohol precursors of Glu-boroPro that are oxidized in vivo (e.g., in the liver) and that have the following structures 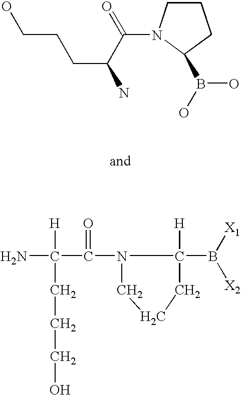
and a boroxine molecule having the following structure 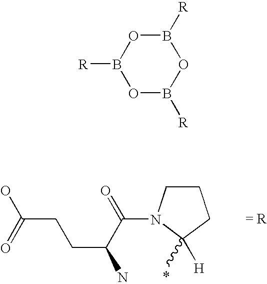
as well as esters of Glu-boroPro and related compounds. Prodrugs of Glu-boroPro also include cyclized versions of the molecule, as discussed above.

Another category of prodrugs includes compounds that are converted to Glu-boroPro by a post-prolyl cleaving enzyme such as DPP-IV. However, the invention is not so limited and other prodrugs are also contemplated including those converted to Glu-boroPro by non-post-prolyl cleaving enzymes. Examples of suitable prodrug moieties are disclosed in U.S. Pat. Nos. 5,462,928 issued Oct. 31, 1995; and 6,100,234 issued Aug. 8, 2000; and published PCT applications WO 91/16339 published Oct. 31, 1991; WO 93/08259 published Apr. 29, 1993; and WO 03/092605, published Nov. 13, 2003, among others.

The length of such prodrug compounds may be 4, 6, 8, 10, 12, 14, 16, 18, 20, 30, 50, 100 or more residues in length (whereby the length includes the glutamic acid and proline residues). Multiples of 3 are also contemplated. The residues may be amino acid in nature (including naturally and non-naturally occurring amino acids). Examples of naturally occurring amino acids are glycine (Gly), and the D- or L-forms of alanine (Ala), valine (Val), leucine (Leu), isoleucine (Ile), phenylalanine (Phe), tyrosine (Tyr), tryptophan (Trp), cysteine (Cys), methionine (Met), serine (Ser), threonine (Thr), lysine (Lys), arginine (Arg), histidine (His), aspartic acid (Asp), glutamic acid (Glu), asparagine (Asn), glutamine (Gln) and proline (Pro). Examples of non-naturally occurring amino acids include but are not limited to 4-hydroxy-proline (Hyp), 5-hydroxy-lysine, norleucine (Nle), 5-hydroxynorleucine (Hyn), 6-hydroxynorleucine, ornithine, cyclohexylglycine (Chg), N-Methylglycine (N-MeGly), N-Methylalanine (N-MeAla), N-Methylvaline (N-MeVal), N-Methylleucine (N-MeLeu), N-Methylisoleucine (N-MeIle), N-Methylnorleucine (N-MeNle), N-Methyl-2-aminobutyric acid (N-MeAbu) and N-Methyl-2-aminopentanoic acid (N-MeNva).

As mentioned above, the specification focuses on boronic acid containing compounds as an exemplary species of agents to be used in the invention. It is to be understood however that other reactive moieties can be used in place of the boronic acid functional group. These include but are not limited to phosphonates such as organo phosphonates and peptidyl (alpha-aminoalkyl) phosphonate esters, fluoroalkylketones, alphaketo amides, alphaketo esters, alphaketo acids, N-peptiolyl-O-acylhydroxylamines, azapeptides, azetidines, fluoroolefins dipeptide isoesters, cyanopyrrolidines, aminoacyl pyrrolidine-2-nitriles and thiazolides such as 4-cyanothiazolidides.

The residues may also be comprised of saccharides, fatty acids, sterols, isoprenoids, purines, pyrimidines, derivatives or structural analogs of the above, peptoids, random bio-oligomers (U.S. Pat. No. 5,650,489), benzodiazepines, diversomeres such as dydantoins, nonpeptidyl peptidomimetics with a beta-D-glucose scaffolding, oligocarbamates, or combinations thereof and the like. Many, if not all, of these compounds can be synthesized using recombinant or chemical library approaches. A vast array of compounds can be generated from libraries of synthetic or natural compounds.

The methods provided herein embraces treatment methods. As used herein, the term “treatment” refers to the administration of one or more therapeutic agent to a subject for the purpose of achieving a medically desirable benefit. Accordingly, “treatment” intends to embrace both “prophylactic” and “therapeutic” treatment methods. Prophylactic treatment methods refer to treatment administered to a subject at risk of developing a glucose-associated condition such as type 2 diabetes (e.g., a prediabetic subject). Therapeutic treatment methods refer to treatment administered to a subject after the diagnosis of such a condition.

A subject shall mean a human or animal including but not limited to a dog, cat, horse, cow, pig, sheep, goat, chicken, rodent e.g., rats and mice, primate, e.g., monkey, and fish or aquaculture species such as fin fish (e.g., salmon) and shellfish (e.g., shrimp and scallops), provided that it would benefit from the methods provided herein. Subjects suitable for therapeutic or prophylactic methods include vertebrate and invertebrate species. Subjects can be house pets (e.g., dogs, cats, fish, etc.), agricultural stock animals (e.g., cows, horses, pigs, chickens, etc.), laboratory animals (e.g., mice, rats, rabbits, etc.), zoo animals (e.g., lions, giraffes, etc.), but are not so limited. In all embodiments, human subjects are preferred. Human subjects can be subjects at any age, including adults, juveniles, infants and fetuses in utero. Pregnant subjects such as pregnant human subjects are also contemplated.

One category of subjects to be treated according to the invention are those that demonstrate impaired glucose tolerance (or glucose intolerance), such as but not limited to subjects having or at risk of developing type 2 diabetes. These subjects generally demonstrate an inability to control glucose levels upon eating, as would a non-diabetic or non-prediabetic “normal” subject. Subjects at risk of developing type 2 diabetes who demonstrate impaired glucose tolerance are considered to be in a prediabetic state. Glucose tolerance can be measured using glucose challenge tests. There are at least two such tests currently available: the Fasting Plasma Glucose Test (FPG) and the Oral Glucose Tolerance Test (OGTT). In human subjects, a FPG blood glucose level between 100-125 mg/dl of blood is indicative of a prediabetic state and an FPG blood glucose level equal to or greater than 126 mg/dl of blood is indicative of diabetes. OGTT measures blood glucose level two hours after ingestion of a glucose-rich drink (which itself occurs after a fasting period). An OGTT blood glucose level between 140-199 mg/dl is indicative of prediabetes, and a level equal to or greater than 200 mg/dl is indicative of diabetes. The presence of glycosylated hemoglobin at levels equal to or greater than 7.0% is also considered an early indicator of the onset of diabetes.

Risk factors for type 2 diabetes include obesity, family history of diabetes, prior history of gestational diabetes, impaired glucose tolerance (as discussed above), physical inactivity, and race/ethnicity. African Americans, Hispanic/Latino Americans, American Indians, and some Asian Americans and Pacific Islanders are at particularly high risk for type 2 diabetes.

Subjects at risk of developing diabetes also may be overweight to the point of being obese. The state of being overweight or obese is defined in terms of the medically recognized body mass index (BMI). BMI equal to a person's body weight (kg) divided by the square of his or her height in meters (i.e., wt/(ht)²). A subject having a BMI of 25 to 29.9 is considered overweight. A subject having a BMI of 30 or more is considered obese.

Symptoms associated with diabetes include but are not limited to frequent urination, excessive thirst, extreme hunger, unusual weight loss, increased fatigue, irritability and blurred vision.

Diabetes is associated with other conditions, many of which result from a diabetic state. These include acute metabolic complications such as diabetic ketoacidosis and hyperosmolar coma, and late complications such as circulatory abnormalities, retinopathy, nephropathy, neuropathy and foot ulcers. A more detailed description of the foregoing terms can be obtained from a number of sources known in the art (see, e.g., Harrison's Principles of Experimental Medicine, 13^{th} Edition, McGraw-Hill, Inc., N.Y.). Thus, the methods of the invention also embrace ameliorating or resolving diabetes-associated conditions such as but not including those recited above.

The compounds of the invention are administered in effective amounts. Generally, an effective amount may vary with the subject's age, condition, and sex, as well as the extent of the disease in the subject (e.g., whether the subject is diabetic or prediabetic) and can be determined by one of skill in the art. The dosage may be adjusted by the individual physician in the event of any complication.

An effective amount typically will vary from about 0.001 μg/kg to about 1000 μg/kg, from about 0.01 μg/kg to about 750 μg/kg, from about 0.1 mg/kg to about 500 μg/kg, from about 1.0 μg/kg to about 250 μg/kg, from about 10.0 μg/kg to about 150 μg/kg in one or more dose administrations daily, for one or several days (depending of course of the mode of administration and the factors discussed above). Other suitable dose ranges include 1 μg to 10000 μg per day, 100 μg to 10000 μg per day, 500 μg to 10000 μg per day, and 500 μg to 1000 μg per day. In some particular embodiments, the amount is less than 10,000 μg per day with a range of 750 μg to 9000 μg per day. In one embodiment, the effective amount for treating or preventing a glucose-associated condition such as type 2 diabetes is an amount that does not stimulate cytokine or chemokine induction by the active agent. Although not intending to be bound by any particular theory, the dose of Glu-boroPro required to stimulate cytokine or chemokine induction may be on the order of 100-fold more than the dose required for treatment according to the methods of the present invention.

As described in greater detail in the Examples, administration of Glu-boroPro leads to, inter alia, inhibition of DPP-IV and to changes in glucose excursion following food intake. The amount of Glu-boroPro required for treatment according to the invention therefore can also be described in terms of the amount of DPP-IV inhibition. For example, the amount of Glu-boroPro required to treat glucose-associated conditions such as diabetes may also be the amount that inhibits at least and preferably more than 90% of serum DPP-IV, as measured by standard DPP-IV activity assays. The amount of Glu-boroPro required to treat glucose-associated conditions such as diabetes may also be the amount that reduces a glucose excursion “area under the curve” by about 40-50% relative to a control or untreated subject profile. The “area under the curve” measurement is demonstrated in the Examples and Figures and is a composite measure of the peak and breadth of the glucose profile in a subject, for example, after food intake. Administration of Glu-boroPro and related compounds can effect a reduction in the glucose peak and/or in the length of time necessary to recover to a normal level of glucose, for example, after food intake.

Unit dosages (i.e., the amount of Glu-boroPro compound present in a single dose such as a tablet, pill, capsule and the like) preferably are comparable to the effective amounts shown above. Unit dosages will depend upon how often the agent is administered, whether it is administered together with a second agent, and the route of administration, among other things. As an example however, if the Glu-boroPro compound is orally administered to a subject once a day in the absence of a second anti-diabetic agent, then the unit dosage can be approximately 100 μg, approximately 200 μg, approximately 300 μg, approximately 400 μg, approximately 500 μg, approximately 600 μg, approximately 700 μg, approximately 800 μg, approximately 900 μg, or approximately 1000 μg. As used herein, approximately means +/−5%. Alternatively, the unit dosage can be in the range of 100-10000 μg, 500-5000 μg, or 500-1000 μg. In some embodiments, the dosage is less than 1000 μg. In other embodiments, the unit dosage range is 750-9000 μg. A unit dosage corresponds to the amount of Glu-boroPro being administered. If Glu-boroPro is provided as a prodrug, then the amount of total compound administered will be in excess of the unit dosage.

As described in greater detail herein, the invention contemplates administration of Glu-boroPro compounds and a second agent such as but not limited to an anti-diabetic agent. In these aspects and embodiments, the dose of the Glu-boroPro compound, the second agent, or both the Glu-boroPro and second agent may be reduced over the dose required when one agent is adminstered alone. For example, the unit dosage of one or both agents may be reduced by a factor of 2, 3, 4, 5, 6, 7, 8, 9, 10, 15, 20, 25, 50, 100 or more relative to the unit dosage required when a single agent is administered. The above teaching similarly applies when the second agent is itself a combination of two or more agents (e.g., such as in the case of the anti-diabetic agent Avandamet). Second agents are generally agents that are used and/or prescribed for the treatment of glucose-associated conditions such as anti-diabetic agents, anti-obesity agents, anti-atherosclerotic agents, anti-retinopathy agents, anti-hyperlipidemia agents, anti-acidosis agents, anti-neuropathy agents, anti-nephropathy agents, anti-hyperglycemia agents, anti-hyperinsulinemia agents, anti-hyperlipidemia agents, anti-hyperlipoproteinemia agents, anti-hypertension agents, anti-inflammatory agents, anti-ulcer agents, and the like. Those of ordinary skill in the art will be familiar with such agents, and in addition reference can be made to Harrison's Principles of Experimental Medicine, 13^{th} Edition, McGraw-Hill, Inc., N.Y. or the Physician's Desk Reference (PDR).

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. Preferably, the agent is administered once a day in order to facilitate patient compliance.

The agents may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks therebetween. Alternatively, the predetermined routine schedule may involve administration on a twice daily basis for the first week, followed by a daily basis for several months, etc. Any particular combination would be covered by the routine schedule as long as it is determined ahead of time that the appropriate schedule involves administration on a certain day.

Preferably, the agents are designed to be delivered with greatest ease to subjects. This may include for example a once a day oral administration, the timing of which is not dependent upon food intake. Thus, for example, the agent can be taken every morning and/or every evening, regardless of when the subject has eaten or will eat.

Glu-boroPro compounds may be administered together with other therapeutic agents, such as those discussed above. As used herein, a therapeutic agent is intended to embrace agents that work therapeutically and/or prophylactically. Depending on the timing and route of administration, the Glu-boroPro compounds and the second therapeutic agent may be administered in the same administration vehicle (e.g., tablet, implant, injectable solution, etc.). Alternatively, the agents may be separately dosed and administered.

Glu-boroPro compounds may be administered substantially simultaneously with the other therapeutic agent. By substantially simultaneously, it is meant that the Glu-boroPro compound is administered to a subject close enough in time with the administration of the other agent so that the two compounds may exert an additive or even synergistic effect. The agents of the invention may be administered or used together with non-drug therapies such as but not limited to non-drug anti-diabetic therapies such as carbohydrate reduced diets.

One therapeutic agent of interest is an anti-diabetic agent. An anti-diabetic agent is an agent that is used in the prevention and/or treatment of prediabetes or diabetes in order to regulate glucose. There are various categories of anti-diabetic agents. These include insulin, peroxisome proliferator-activated receptor-γ (PPARγ) agonists, inhibitors of hepatic glucose production, stimulators of insulin release from pancreas, glucosidase inhibitors, and incretin and incretin analogues.

Insulin includes rapid-acting forms, intermediate-acting forms, and long-acting forms. Basal insulin, using long-acting insulins, can be injected once or twice a day. Bolus (or mealtime) insulin, using rapid-acting insulins, covers mealtime carbohydrates and corrects the current glucose level.

Rapid-acting forms of insulin include Insulin lispro rDNA origin: HUMALOG® (1.5 mL, 10 mL, Eli Lilly and Company, Indianapolis, Ind.), HUMALOG® Mix 75/25-Pen, Insulin Injection (Regular Insulin) form beef and pork (regular ILETIN® I, Eli Lilly], human: rDNA: HUMULIN® R (Eli Lilly), HUMULIN® 50/50, HUMULIN® 70/30, NOVOLIN® R (Novo Nordisk, New York, N.Y.), NOVOLIN(® 70/30 Human Insulin, NOVOLIN® 70/30 PenFill, NOVOLIN® Innolet, Semisynthetic: VELOSULIN® Human (Novo Nordisk), rDNA Human, Buffered: VELOSULIN® BR, pork: regular Insulin (Novo Nordisk), purified pork: Pork Regular ILETIN® II (Eli Lilly), Regular Purified Pork Insulin (Novo Nordisk), and Regular (Concentrated) ILETIN® II U-500 (500 units/mL, Eli Lilly); NovoLog Mix 70/30.

Intermediate-acting forms of insulin include Insulin Zinc Suspension, beef and pork: LENTE® ILETIN® I (Eli Lilly), Human, rDNA: HUMULIN® L (Eli Lilly), HUMULIN N, HUMULIN® N pen, NOVOLIN® L (Novo Nordisk), NOVOLIN N Human Insulin, NOVOLIN® N PenFill; purified pork: LENTE® ILETIN® II (Eli Lilly), Isophane Insulin Suspension (NPH): beef and pork: NPH ILETIN® I (Eli Lilly), Human, rDNA: HUMULIN® N (Eli Lilly), NOVOLIN® N (Novo Nordisk), purified pork: Pork NPH Iletin® II (Eli Lilly), NPH-N (Novo Nordisk).

Long-acting forms of insulin include Insulin zinc suspension, extended (ULTRALENTE®, Eli Lilly), human, rDNA: HUMULIN® U (Eli Lilly), Lantus Injection.

PPARγ agonists function as insulin-sensitizing agents that primarily enhance peripheral glucose utilization. PPARγ is a nuclear receptor that regulates transcription of insulin-responsive genes that in turn control glucose production, transport, and utilization and regulate fatty acid metabolism.

An example of PPARγ agonists is thiazolidinediones which include Avandamet (combination of rosiglitazone and metformin), rosiglitazone (Avandia), pioglitazone (Actos), troglitazone (Rezulin), (S)-((3,4-dihydro-2-(phenyl-methyl)-2H-1-benzopyran-6-yl)methyl-thiazolid-ine-2,4-dione (englitazone), 5-{[4-(3-(5-methyl-2-phenyl-4-oxazolyl)-1-oxo- propyl)-phenyl]-methyl}-thiazolidine-2,4-dione (darglitazone), 5-{[4-(1-methyl-cyclohexyl)methoxy)-phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy)]benzyl}-thiazolidine-2,4-dione (BM-13.1246), 5-(2-naphthylsulfonyl)-thiazolidine-2,-4-dione (AY-31637), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-y-1)ethoxy)phenylmethyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)-ethoxy)phenyl]methyl}-thiazolidine-2,-4-dione (rosiglitazone), 5-{[4-(2-(5-ethyl-2-pyridyl)ethoxy)phenyl]-methyl-}thiazolidine-2,4-dione (pioglitazone), 5-{[4-((3,4-dihydro-6-hydroxy-2,5,-7,8-tetramethyl-2H-1-benzopyran-2-yl)methoxy)-phenyl]-methyl}-thiazolidine-2,4-dione (troglitazone), 5-[6-(2-fluoro-benzyloxy)-naphthalen-2-ylmethyl-]-thiazolidine-2,4-dione (MCC555), 5-{[2-(2-naphthyl)-benzoxazol-5-yl]-methyl}thiazolidine-2,4-dione (T-174) and 5-(2,4-dioxothiazolidin-5-ylmethyl)-2-methoxy-N-(4-trifluoromethyl-benzyl)benzamide (KRP297).

Another example of a PPAR γ agonist is natural prostaglandin D(2) (PGD(2)) metabolite, 15-deoxy-Delta(12, 14)-prostaglandin J(2) (15d-PGJ(2)).

Inhibitors of hepatic glucose production act primarily by decreasing hepatic glucose production, decreasing intestinal absorption of glucose and increasing peripheral glucose uptake and utilization. They can function as anti-hyperglycemic agents thereby lowering both basal and postprandial plasma glucose levels. An example of this category of agents is biguanides. Examples of biguanides include metformin (GLUCOPHAGE), Avandamet tablets (metformin combination tablet), Glucovance tablets, and Metaglip tablets.

Stimulators of insulin release from the pancreas act by a mechanism that is unclear, at least for long-term administration effect. When chronically administered, the blood glucose lowering effect of these agents persists despite a gradual decline in insulin secretory response. Extra-pancreatic effects may play a role in the mechanism of action. Examples of this category of agents are sulfonylureas and meglitinides. First-generation sulfonylureas include acetohexamide (DYMELOR), chlorpropamide (DIABINESE) and tolbutamide (ORINASE, RASTINON). Second-generation sulfonylureas include glipizide (GLUCOTROL, GLUCOTROL XL), glyburide (DIABETA; MICRONASE; GLYNASE) and glimepiride (AMARYL). Other sulfonylureas include glisoxepid (PRO-DIABAN), glibenclamide (AZUGLUCON), glibomuride (GLUBORID), tolazamide, carbutamide, gliquidone (GLURENORM), glyhexamide, phenbutamide, tolcyclamide, gliclazide (DIAMICRON).

Meglitinides close ATP-dependent K+ channels in β-cell membrane (selectively vs. heart and skeletal muscle), thereby depolarizing β-cells with consequent opening of Ca2+ channels. The resultant increased Ca²⁺ influx induces insulin secretion. Examples of meglitinides include Repaglinide (PRANDIN) and nateglinide (STARLIX).

Glucosidase inhibitors act by reversibly inhibiting membrane bound intestinal α-glucoside hydrolase enzymes. These enzymes hydrolyze oligosaccharides and disaccharides to glucose in the brush border of the small intestine. Pancreatic α-amylase, which hydrolyzes complex to oligosaccharides in lumen of small intestine, is also inhibited. The enzyme inhibition delays glucose absorption and lowers postprandial hyperglycemia. Examples of alpha-glucosidase inhibitors include Acarbose (PRECOSE, GLUCOBAY), Miglitol (GLYSET, DIASTABOL), and voglibose. Acarbose is 4″,6″-dideoxy-4″-[(1S)-(1,4,6/5)-4,5,6-trihydroxy-3-hydroxymethyl-2-cyclo-hexenylamino}maltotriose (U.S. Pat. No. 4,062,950 and EP 0 226 121).

Incretins and incretin analogues can be used as anti-diabetic agents. These include GLP-1, GIP and their analogues. Analogues of glucagon like peptide-1 (GLP-1) include EXENATIDE (synthetic exendin-4) and EXENATIDE LAR (long acting release).

Other anti-diabetic agents include Buformin; Butoxamine Hydrochloride; Camiglibose; Ciglitazone; Englitazone Sodium; Darglitazone Sodium; Etoformin Hydrochloride; Gliamilide; Glicetanile Gliclazide Sodium; Gliflumide; Glucagon; Glymidine Sodium; Glyoctamide; Glyparamide; Linogliride; Linogliride Fumarate; Methyl Palmoxirate; Palmoxirate Sodium; Pirogliride Tartrate; Proinsulin Human;; Seglitide Acetate; Tolpyrramide; Zopolrestat.

Further anti-diabetic agents are described in detail in U.S. Patents: U.S. Pat. Nos. 6,121,282, 6,057,343, 6,048,842, 6,037,359, 6,030,990, 5,990,139, 5,981,510, 5,980,902, 5,955,481, 5,929,055, 5,925,656, 5,925,647, 5,916,555, 5,900,240, 5,885,980, 5,849,989, 5,837,255, 5,830,873, 5,830,434, 5,817,634, 5,783,556, 5,756,513, 5,753,790, 5,747,527, 5,731,292, 5,728,720, 5,708,012, 5,691,386, 5,681,958, 5,677,342, 5,674,900, 5,545,672, 5,532,256, 5,531,991, 5,510,360, 5,480,896, 5,468,762, 5,444,086, 5,424,406, 5,420,146, RE34,878, 5,294,708, 5,268,373, 5,258,382, 5,019,580, 4,968,707, 4,845,231, 4,845,094, 4,816,484, 4,812,471, 4,740,521, 4,716,163, 4,695,634, 4,681,898, 4,622,406, 4,499,279, 4,467,681, 4,448,971, 4,430,337, 4,421,752, 4,419,353, 4,405,625, 4,374,148, 4,336,391, 4,336,379, 4,305,955, 4,262,018, 4,220,650, 4,207,330, 4,195,094, 4,172,835, 4,164,573, 4,163,745, 4,141,898, 4,129,567, 4,093,616, 4,073,910, 4,052,507, 4,044,015, 4,042,583, 4,008,245, 3,992,388, 3,987,172, 3,961,065, 3,954,784, 3,950,518, 3,933,830, the disclosures of which are incorporated herein by reference.

The invention also contemplates the use of a second agent that is also a DPP-IV inhibitor. These include but are not limited to alanyl pyrrolidine, isoleucyl thiazolidine and O-benzoyl hydroxylamine.

Anti-diabetic agents also include combinations of anti-diabetic agents, many of which are commercially available. These include ACTOS(R) (pioglitazone HCl) in combination with a sulfonylurea, metformin or insulin.

Table 1 shows a list of anti-diabetic agents used singly or in combination.

TABLE 1
Anti-diabetic drug categories

| | Proprietary | |
|---|---|---|
| Category | drug trade name | Anti-diabetic agents in drug |
| Biguanides and | Avandamet | Rosiglitazone maleate |
| combinations | | (thiazolidinedione) + |
| | | metformin HCl (biguanide) |
| | Glucovance | Glyburide (sulphonylurea) + |
| | | metformin HCl (biguanide) |
| | Metaglip | Glipizide (sulphonylureas) + |
| | | metformin HCl (biguanide) |
| Glucosidase | Glyset | Miglitol (oral α-glucosidase |
| inhibitors | | inhibitor) |
| | Precose | Ascarbose (oral α- |
| | | glucosidase inhibitor) |
| Meglitinides | Prandin | Repaglinide (oral |
| | | meglitinide) |
| | Starlix | Nateglinide (oral meglitinide) |
| Sulfonylurea | Amaryl | Glimepiride (oral |
| | | sulfonylurea) |
| | Diaβeta | Glyburide (oral sulfonylurea) |
| | Diabinese | Chlorpropamide (oral |
| | | sulfonylurea) |
| | Glucotrol | Glipizide (oral sulfonylurea) |
| Thiazolidinediones | Actos | Pioglitazone HCl (oral |
| | | thiazolidinedione) |
| | Avandia | Rosiglitazone maleate (oral |
| | | thiazolidinedione) |

Anti-inflammatory agents are agents that reduce inflammation locally or systemically in a subject. Examples of anti-inflammatory agents include Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

A variety of administration routes are available. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular or infusion. Intravenous or intramuscular routes are not particularly suitable for long-term therapy and prophylaxis. They could, however, be preferred in emergency situations. Oral administration is a generally preferred mode of administration because of the convenience to the patient.

When used in vivo, the agents are formulated as pharmaceutical compositions or preparations. In general, a pharmaceutical composition comprises the agent(s) and a pharmaceutically-acceptable carrier. As used herein, a pharmaceutically-acceptable carrier means a non-toxic material that does not interfere with the effectiveness of the biological activity of the agents of the invention.

Pharmaceutically-acceptable carriers include diluents, fillers, salts, buffers, stabilizers, solubilizers and other materials which are well-known in the art. Exemplary pharmaceutically-acceptable carriers for peptides in particular are described in U.S. Pat. No. 5,211,657. Such preparations may routinely contain salt, buffering agents, preservatives, compatible carriers, and optionally other therapeutic or prophylactic agents. When used in medicine, the salts should be pharmaceutically acceptable, but non-pharmaceutically-acceptable salts may conveniently be used to prepare pharmaceutically-acceptable salts thereof and are not excluded from the scope of the invention. Such pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compositions of the invention may be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

Preferably, at least the Glu-boroPro compounds are formulated for oral administration. Compositions suitable for oral administration may be presented as discrete units, such as capsules, tablets, lozenges, each containing a predetermined amount of the active agent. Other compositions include suspensions in aqueous liquids or non-aqueous liquids such as a syrup, elixir or an emulsion.

For oral administration, the agents can be formulated readily by combining the active compound(s) with pharmaceutically-acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

The agents may be administered directly to a tissue. Preferably, the tissue is one affected by the diabetic or prediabetic state and is likely to respond beneficially to the agent an example is the pancreas or tissue surrounding the pancreas. Direct tissue administration may be achieved by direct injection. If the agents are administered multiple times, the compositions may be administered via different routes. For example, the first (or the first few) administrations may be made directly into the affected tissue while later administrations may be systemic.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Other delivery systems can include time-release, delayed release or sustained release delivery systems. Such systems can avoid repeated administrations of the agent, increasing convenience to the subject and the physician. Many types of release delivery systems are available and known to those of ordinary skill in the art. They include polymer based systems and non-polymer based systems such as lipids including sterols such as cholesterol, cholesterol esters and fatty acids or neutral fats such as mono-, di-, and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the agent is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for prophylactic treatment of subjects. Long-term release, as used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In some embodiments, the delivery vehicle is a biocompatible microparticle or implant that is suitable for implantation into the mammalian recipient. Exemplary bioerodible implants that are useful in accordance with this method are described in PCT International Application No. PCT/US/03307 (Publication No. WO 95/24929, entitled “Polymeric Gene Delivery System”, claiming priority to U.S. patent application Ser. No. 213,668, filed Mar. 15, 1994). PCT/US/0307 describes a biocompatible, preferably biodegradable polymeric matrix for containing a biological macromolecule. The polymeric matrix may be used to achieve sustained release of the agent in a subject. In accordance with one aspect of the instant invention, the agent described herein may be encapsulated or dispersed within the biocompatible, preferably biodegradable polymeric matrix disclosed in PCT/US/03307. The polymeric matrix preferably is in the form of a microparticle such as a microsphere (wherein the agent is dispersed throughout a solid polymeric matrix) or a microcapsule (wherein the agent is stored in the core of a polymeric shell). Other forms of the polymeric matrix for containing the agent include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used. The polymeric matrix composition can be selected to have both favorable degradation rates and also to be formed of a material which is bioadhesive, to further increase the effectiveness of transfer. The matrix composition also can be selected not to degrade, but rather, to release by diffusion over an extended period of time.

Both non-biodegradable and biodegradable polymeric matrices can be used to deliver the agents to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multivalent ions or other polymers.

In general, the agents of the invention may be delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, poly-vinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polymers of lactic acid and glycolic acid, polyanhydrides, poly(ortho)esters, polyurethanes, poly(butic acid), poly(valeric acid), and poly(lactide-cocaprolactone) and polyvinylpyrrolidone.

Examples of biodegradable polymers include natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Examples of non-biodegradable polymers include ethylene vinyl acetate, poly(meth)acrylic acid, polyamides, copolymers and mixtures thereof.

Bioadhesive polymers of particular interest include bioerodible hydrogels described by H. S. Sawhney, C. P. Pathak and J. A. Hubell in Macromolecules, 1993, 26, 581-587, the teachings of which are incorporated herein, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The invention further provides kits that comprise the agents of the invention and optionally instructions of use thereof. The agents may be present in oral forms such as tablets, pills, capsules, caplets and the like. The agents may be provided in a one a day dispensing unit such as a blister pack or dial pack type dispenser, preferably with days of the week or day of the month (e.g., 1, 2, 3, 4, etc.) (and doses per day, where applicable) printed on the dispenser. For example, if the agents are to be administered every other day or twice (or more) a day, the dispensing unit can be modified accordingly, with no more than routine reconfiguration, known in the art. The kit may further contain a second agent such as a second anti-diabetic agent, either formulated together with the Glu-boroPro compound of the invention or formulated separately. The unit dosages provided in each form (e.g., tablet, pill, capsule, etc.) will depend upon whether the Glu-boroPro compound is used together with or in the absence of a second agent. The kit may optionally comprise a housing such as a box or bag. Instructions for use may be supplied separately from the dispensing unit or housing or they may be imprinted on one or both.

The following examples are provided to illustrate specific instances of the practice of the present invention and are not intended to limit the scope of the invention. As will be apparent to one of ordinary skill in the art, the present invention will find application in a variety of compositions and methods.

### EXAMPLES

### Example 1

This example illustrates the kinetics of in vitro DPP-IV inhibition by Glu-boroPro. The enzyme inhibitory activity of Glu-boroPro is compared with that of other amino boronic dipeptides in in vitro assays with isolated DPP-IV.

### Materials and Methods

Production of Soluble Recombinant Human DPP-IV

Based on information on the N-terminus of serum DPP-IV (15), a truncated DPP-IV was engineered in which a signal/leader sequence was joined to the residue in DPP-IV corresponding to the N-terminus of serum DPP-IV to allow secretion. The cDNA encoding the desired truncated human DPP-IV dimer enzyme was engineered into the mammalian secretion vector pSecTag2 (Cat# V900-20, InVitrogen Corporation). The vector, available in A, B or C versions, representing three possible phases for gene fusion, contained an immunoglobulin-kappa light chain secretion signal followed by a selection of restriction sites for gene insertion. The fusion required engineering a restriction site upstream of the chosen fusion amino acid in the 5′ end of the DPP-IV dimer enzyme nucleic acid in phase with the chosen restriction site (Sfi I) in the vector secretion sequence. The chosen fusion amino acid in the 5′ end of the DPP-IV (Ser39) was 3′ of the trans-membrane anchoring domain. The pSecTag2 version B and its Sfi I restriction site was chosen for the fusion because it minimizes the additional N-terminal, vector-encoded residues in the mature secreted protein.

| Sequence of N-terminus of serum hDPP-IV (15): | | |
|---|---|---|
| hDPP-IV: | MKTPWKVLLGLLGAAALVTIITVPVVLLNKGTDDATADSRKTYTLTDYLKN-- | (SEQ ID NO: 1) |
| Serum DPP-IV: | SRKTYTLTDYLKN-- | (SEQ ID NO: 2) |
| | SRKTYTLTDYLKN-- | (SEQ ID NO: 3) |

Construction of the fusion was as follows. First, total RNA was isolated from the Caco-2 colorectal carcinoma cell line (ATCC HTB-37) by standard Trizol/phenol/chloroform methodology. The purified RNA (approx. 2.5 μg in a 20 μl reaction) was used to make cDNA using oligo-dT primer and a commercial reverse transcription (RT) kit (InVitrogen). An aliquot (2 μl) of the RT reaction was used to amplify by polymerase chain reaction (PCR), a truncated coding region of human DPP-IV dimer enzyme cDNA corresponding to nucleotide 225-2408 approximately of wild type DPP-IV dimer enzyme (GenBank Accession number NM_{—}001935). The Taq DNA polymerase-mediated PCR was performed with primers: Sfi-DPP-IV (5′ GTAGTCGGCC CAGCCGGCC AGTCGCAAAA CTTACACTCT AACTGATTAC TTAAAAAAT 3′, SEQ ID NO: 4) containing a Sfi I restriction site (underlined) and primer DPP4-R 5′ GTCGGAGCGG CCGCCTAAGG TAAAGAGAAA CATTGTTTTA TGAAGTG 3′ (SEQ ID NO: 5) containing a Not I site (underlined). The following thermal cycler program was used: 94° C. for 45 sec. initial denaturation, then 30 cycles of 94° C., 10 sec.; 48° C., 6 sec.; 60° C., 4 min; followed by 5-min. extension at 72° C. after cycling. The resultant PCR product was cleaved with restriction enzymes SfiI for 25 min at 50° C., then 1 hr with NotI at 37° C. The approx. 2.2 kb fragment was isolated from an agarose gel using standard procedures and ligated to pSecTag2-B vector (InVitrogen, Cat. # V900-20) fragments (5.6 kb) that had been similarly prepared using the same enzymes. After transformation into bacteria under standard conditions and screening of colonies, those with correct properties were sequenced to ensure the correct fusion junction and absence of PCR-induced mutations, giving a plasmid designated #135 which was designated wild-type DPP-IV. The resulting plasmid #135, contained DPP-IV truncated, as described above, and fused to a plasmid encoded immunoglobulin Kappa chain secretion sequence under control of the CMV promoter (U.S. Pat. Nos. 5,168,062 and 5,383,839) with a 3′ bovine growth hormone polyadenylation sequence (U.S. Pat. No. 5,122,458). The N-terminus of the final mature amino acid sequence of mature (cleaved) secreted product contains 6 amino acids having a sequence of DAAQPR (SEQ ID NO:6) or DAAQPA (SEQ ID NO:7), fused to the truncated DPP-IV sequence starting at Ser39, the first 13 amino acids of which are SRKTYTLTDYLKN (SEQ ID NO:2).

DNA from the plasmid encoding DPP-IV dimer enzyme was prepared on an approximately 400 μg scale from overnight 30 ml cultures in Luria broth with 100 μg ampicillin per ml using a commercial kit (Qiagen Maxiprep Kit). Ten (10) μg of DNA and 30 μl of Lipofectamine 2000 transfection reagent (InVitrogen Corporation) were used to transiently transfect 293T cells in 10 cm diameter tissue culture plates using the manufacturer's protocol. Cells were grown to >70% confluent in Freestyle 293 Expression Medium (InVitrogen Corporation) containing 2.5% fetal calf serum and standard antibiotics penicillin and streptomycin. Antibiotic-free medium was used for the initial 18-24 hours of transfection, after which serum-free medium with antibiotics was employed. Culture supernatant containing the secreted recombinant enzyme was harvested 6-18 hours later and again 24 hours after addition of fresh serum-free medium and was stored 4° C.

In vitro Assay of Enzymatic Activity of Recombinant Soluble DPP-IV and Inhibition by Amino Boronic Pipeptides

The assay reaction mixture consisted of 135 μl 50 mM HEPES/Na buffer pH 7.6, 140 mM NaCl, 10-15 μl enzyme-containing culture supernatant, dipeptide substrate Ala-Pro-(7-amino-4-trifluoromethyl coumarin) (Ala-Pro-AFC; Enzyme System Products, Dublin, Calif.) at typically 0.1-1 mM added from a 100 or 400 mM stock in dimethyl formamide. The reaction mixture was incubated at room temperature, and production of the fluorescent AFC product was measured in a fluorometer (excitation, 400 nm; emission 505 nm), either by continuous monitoring or after termination with a one to one-tenth volume of 1-M sodium acetate, pH 4.5. Fluorometric reading were made with a Molecular Dynamics Spectra Max GeminiXS capable of reading 96-well microtiter plates. The inhibitory activity of amino boronic dipeptides was investigated by preincubation of assay reaction mixtures with varying concentrations of each compound for 10 minutes before the addition of the substrate Ala-Pro-AFC. The completed reaction mixtures were then incubated for 3 minutes, 10 minutes, 78 minutes, or 16 hours and read fluorometrically.

Results:

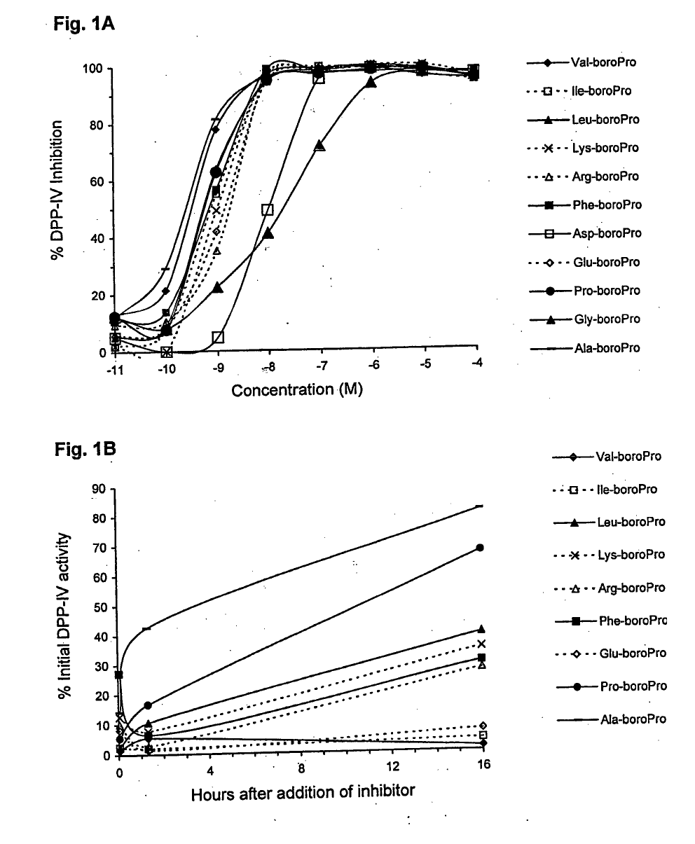

FIG. 1A illustrates an in vitro dose-response comparison of soluble recombinant DPP-IV enzymatic inhibition by Val-boroPro, Ile-boroPro, Leu-boroPro, Lys-boroPro, Arg-boroPro, Phe-boroPro, Asp-boroPro, Glu-boroPro, Pro-boroPro, Gly-boroPro, and Ala-boroPro. All the amino boronic dipeptides except Asp-boroPro and Gly-boroPro exhibited IC₅₀ (inhibitory concentration 50%, i.e., the concentration of compound required to inhibit enzymatic activity by 50% of control activity) values in the low to sub nanomolar range when DPP-IV was preincubated for 10 minutes with each amino boronic dipeptide before addition of the substrate, Ala-Pro-AFC, and fluorometric measurement after further incubation for 10 minutes. In a separate experiment, comparison of DPP-IV inhibition assayed at 3 minutes, 78 minutes and 16 hours after the addition of Ala-Pro-AFC revealed that preincubation with Glu-boroPro, Val-boroPro and Ile-boroPro resulted in sustained DPP-IV inhibition (>10% of initial DPP-IV activity) for up to 16 hours, whereas inhibition by Ala-boroPro, Pro-boroPro, Leu-boroPro, Lys-boroPro, Phe-boroPro, and Arg-boroPro appeared to be more rapidly reversible (FIG. 1B).

### Example 2

This example illustrates the kinetics of serum DPP-IV inhibition by Glu-boroPro in vivo in mice. The enzyme inhibitory activity of Glu-boroPro is compared with that of other amino boronic dipeptides in in vitro assays with isolated DPP-IV.

### Materials and Methods

Assay of Serum DPP-IV Inhibition In Vivo

Varying doses (0.02, 0.2, 2.0, 20.0 μg/mouse) of Glu-boroPro dissolved in normal saline or the saline vehicle alone were administered to BALB/c mice by oral gavage. Each mouse received a single administration of Glu-boroPro or saline, and blood samples were withdrawn from mice 2 hours later. In studies of the duration of DPP-IV inhibition after administration of 5 or 10 μg/mouse of Glu-boroPro, blood samples were withdrawn at 1, 2, 4, 6, 11, 24, 26 and 48 hours after Glu-boroPro or saline administration. DPP-IV activity was determined by reaction of 10 μl serum with 90 μl of 0.11 mM Ala-Pro-AFC (Enzyme System Products, Dublin, Calif.) in 50 mM HEPES/Na buffer pH 7.6, 140 mM NaCl. Assays were incubated for 30 minutes, stopped and fluorometric measurements made as described in Example 1. Serum DPP-IV activity was expressed as a percentage of the baseline activity in control mice receiving saline, or the activity in mice prior to administration of Glu-boroPro.

Results:

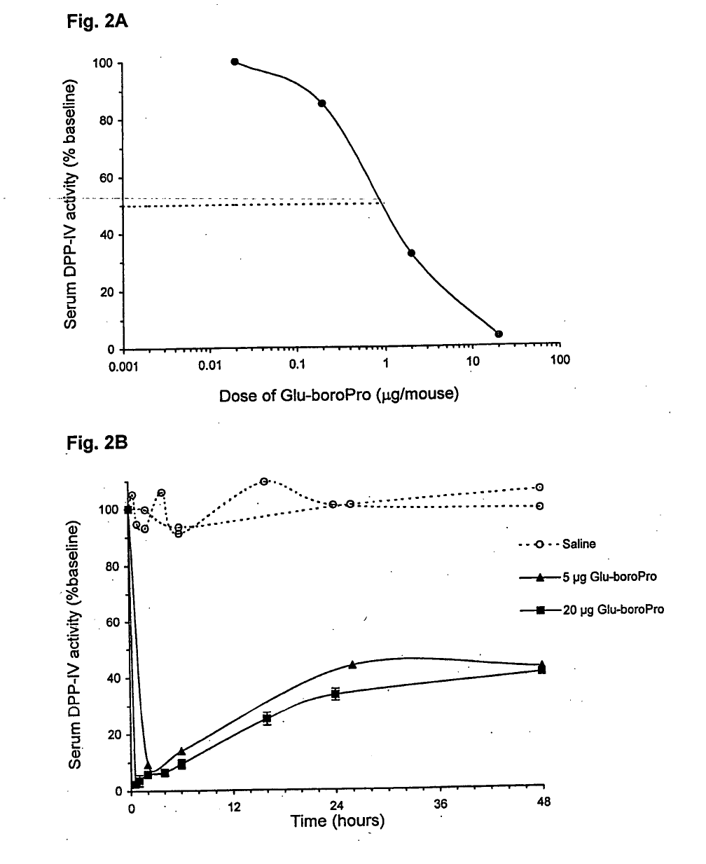

FIG. 2A illustrates a typical dose response for the inhibition of DPP-IV activity in the serum of BALB/c mice administered Glu-boroPro orally. In this experiment, the ID₅₀ (inhibitory dose 50%, i.e., the dose required to reduce serum DPP-IV activity by 50% of baseline in control animals) was determined to be a 1 μg dose of Glu-boroPro per mouse. The duration of serum DPP-IV inhibition after a single oral administration of 5 μg or 20 μg Glu-boroPro per mouse was determined in two experiments (FIG. 2B). The data indicate that greater than 80% of DPP-IV inhibition persisted until at least 6 hours after Glu-boroPro administration.

### Example 3

This example illustrates that, unlike the amino boronic peptides Val-boroPro, Ile-boroPro and Leu-boroPro, Glu-boroPro does not appear to stimulate cytokine production by cultured human bone marrow stromal cells in vitro, as indicated by measurement of the levels of granulocyte colony stimulating factor (G-CSF) in culture supernatants. G-CSF was assayed because it was previously shown to be an indicator of increased levels of cytokines in stromal cell cultures stimulated with Val-boroPro (16).

### Materials and Methods

Human Bone Marrow Stromal Cell Cultures

Samples of normal human bone marrow were purchased from Cambrex Bioproducts (Walkersville, Md.) and mononuclear cells were purified over Ficoll-Hypaque (Nycomed, Oslo, Norway). Human stromal layers were established by seeding 4×10⁷ mononuclear cells into T75 flasks (Corning) containing 20 ml MyeloCult medium (Stem Cell Technologies, Vancouver, BC) supplemented with 10⁻⁶ M hydrocortisone (Sigma) and incubation at 37° C. in 100% humidified 5% CO₂ in air. After one week, half the medium was exchanged, and the cultures incubated for approximately one week more, after which time, a semi-confluent cell layer was formed. Stromal cells were harvested by trypsinization using standard technique and 10⁵ cells/well were seeded in multi-well plates in 1 ml of fully supplemented DMEM (InVitrogen, Carlsbad, Calif.). Val-boroPro, Ile-boroPro, Leu-boroPro or Glu-boroPro were each added to triplicate multiwell cultures at concentrations of 1, 10, 100, 10³ and 10⁴ nM. Multiwell cultures without the addition of amino boronic dipeptides served as controls.

Assay of G-CSF Supernatant Levels in Stromal Cell Cultures

After incubation of multi-well cultures for 24 hours, supernatants were harvested. Supernatant concentrations of human G-CSF were determined by Quantikine enzyme-linked immunosorbent assay (ELISA; R&D Systems, Minneapolis, Minn.) according to the manufacturer's instructions. ELISA was performed in duplicate for each sample. G-CSF concentrations were compared between cultures containing amino boronic dipeptide and control cultures. The effect of each amino boronic dipeptide on the level of supernatant G-CSF was determined by calculating a stimulation index (SI): SI=(mean G-CSF concentration in culture with amino boronic dipeptide)/(mean G-CSF concentration in control culture).

Results

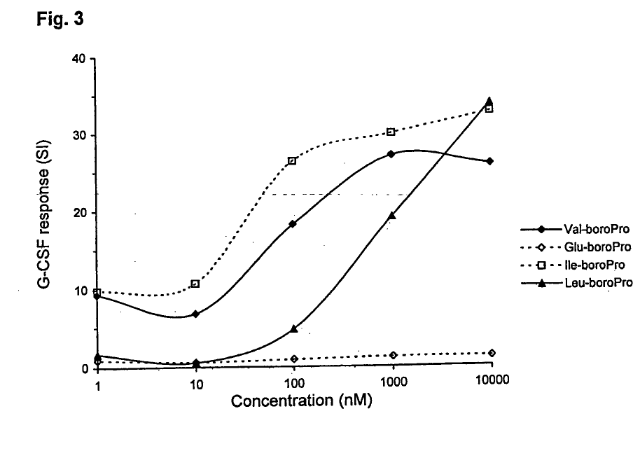

FIG. 3 illustrates the in vitro dose responses of human bone marrow stromal cell cultures to the addition of Val-boroPro, Ile-boroPro, Leu-boroPro or Glu-boroPro as determined by supernatant levels of G-CSF. The SI revealed that, unlike Val-boroPro, Ile-boroPro and Leu-boroPro, Glu-boroPro did not appear to stimulate increased levels of G-CSF in culture supernatants after incubation in vitro for 24 hours.

### Example 4

This example illustrates that, unlike the amino boronic peptides Val-boroPro, Ile-boroPro and Leu-boroPro, Glu-boroPro does not appear to stimulate increased levels of serum KC/CXCL1 in BALB/c mice in vivo at doses that optimally inhibit serum DPP-IV activity. Serum KC/CXCL1 was assayed because it was previously shown to be an indicator of increased levels of cytokines and chemokines in the serum of mice administered Val-boroPro (16, 17).

### Materials and Methods

Assay of Serum DPP-IV Inhibition and KC/CXCL1 Levels In Vivo

Varying doses (0.2, 2.0, 20.0 and 200.0 μg/mouse) of Val-boroPro, Ile-boroPro, Leu-boroPro or Glu-boroPro dissolved in normal saline or the saline vehicle alone were administered to BALB/c mice by oral gavage. Each mouse received a single administration of each amino boronic dipeptide or saline, and blood samples were withdrawn from mice 2 hours later.

Serum DPP-IV activity was determined by reaction of a 10 μl volume of serum with 0.1 mM Ala-Pro-AFC (Enzyme System Products, Dublin, Calif.) in a 100 μl volume of 50 mM HEPES/Na buffer pH 7.6, 140 mM NaCl. Assays were incubated for 30 min, stopped with 1-M sodium acetate, and fluorometric measurements were made as described in Example 1. Serum DPP-IV activity was expressed as fluorescent units (FU).

Serum concentration of mouse KC/CXCL1 was determined by Quantikine enzyme-linked immunosorbent assay (ELISA; R&D Systems, Minneapolis, Minn.) according to the manufacturer's instructions. ELISA was performed in duplicate for each sample.

Results

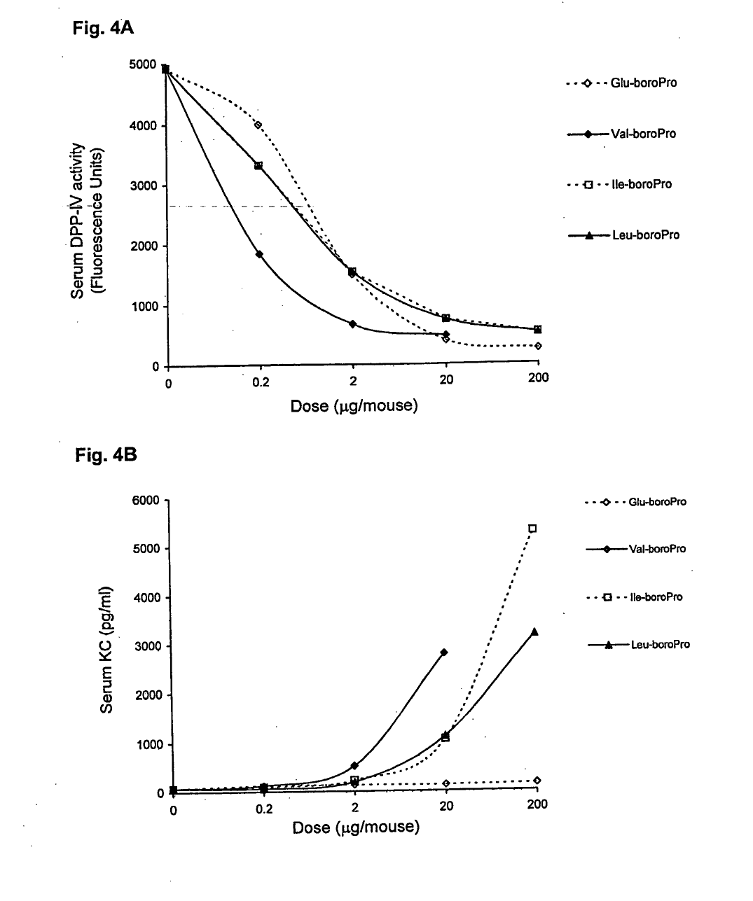

FIG. 4A illustrates typical in vivo dose responses for the inhibition of DPP-IV activity in the serum of BALB/c mice 2 hours following a single oral administration of Val-boroPro, Ile-boroPro, Leu-boroPro or Glu-boroPro. FIG. 4B illustrates the ability of Val-boroPro, Ile-boroPro and Leu-boroPro to stimulate increased serum levels of KC/CXCL1 in a dose-dependent manner. In marked contrast, Glu-boroPro failed to affect serum levels of KC/CXCL1 at any of the doses tested. The data of FIGS. 4A and 4B were obtained from the same serum samples collected from the mice after administration of the amino boronic dipeptides, thereby clearly demonstrating that the 20 and 200 μg doses of Glu-boroPro that maximally inhibited serum DPP-IV activity did not elicit a serum KC/CXCL1 response.

### Example 5

This example illustrates that among the amino boronic dipeptides shown to be potent inhibitors of DPP-IV in vitro, as indicated by IC₅₀ values in the low- to sub-nanomolar range (see Example 1), Glu-boroPro is distinguished by an ID₅₀ of 95 μg/kg and the lowest toxicity in Lewis rats [maximal tolerated dose (MTD) of 15 mg/kg administered as a single dose].

### Materials and Methods

Serum DPP-IV Inhibition and Observation of Acute Toxicity of Amino Boronic Dipeptides in Lewis Rats

Groups of 2-3 rats were administered single escalating doses of Val-boroPro, Ile-boroPro, Met-boroPro, Leu-boroPro, Thr-boroPro, Gln-boroPro, Ala-boroPro, Lys-boroPro, Pro-boroPro, Arg-boroPro, Ser-boroPro or Glu-boroPro. Doses were initially increased in steps of 10 or 20 μg/kg in order to span a dose range of 10 to 200 μg/kg and in steps of 50 to 200 μg/kg for a higher dose range of 200-2000 μg/kg. Utilizing the DPP-IV assay described in example 2, serum DPP-IV activity was determined after 2 hours in rats administered Glu-boroPro and Val-boroPro. The health of the rats was monitored by visual inspection for a period of 5 days, thereby allowing the maximal tolerated dose (MTD) to be recorded for each amino boronic dipeptide as the dose level immediately beneath the dose that caused the rats to become moribund. After observations were completed or at the onset of a moribund state, humane euthanasia was performed by asphyxiation in 100% CO₂.

Results

The MTD obtained in acute toxicity studies in Lewis rats (Table 2) illustrate a range from 20 μg/kg for Val-boroPro to 15 mg/kg for Glu-boroPro. Interestingly, the dose responses for serum DPP-IV inhibition indicated ID₅₀ values of 9 μg/kg for Val-boroPro 5 and 95 μg/kg for Glu-boroPro, following a single oral administration. Consequently, Glu-boroPro was only ˜10-fold less potent than Val-boroPro as an inhibitor of serum DPP-IV and yet was 750-fold less toxic.

TABLE 2
Maximum tolerated doses after acute (single dose) administration
of amino boronic dipeptides to Lewis rats

| Compound | MTD¹ (μg/kg) | ID₅₀² (μg/kg) |
|---|---|---|
| Val-boroPro | 20 | 9 |
| Ile-boroPro | 120 | NT³ |
| Met-boroPro | 160 | NT |
| Leu-boroPro | 200 | NT |
| Thr-boroPro | 800 | NT |
| Gln-boroPro | ≧800 | NT |
| Ala-boroPro | ≧2,000 | NT |
| Lys-boroPro | ≧2,000 | NT |
| Pro-boroPro | ≧2,000 | NT |
| Arg-boroPro | ≧2,000 | NT |
| Ser-boroPro | 4,000 | NT |
| Glu-boroPro | 15,000 | 95 |

¹Maximum tolerated dose
²Inhibitory dose 50%: i.e. dose causing a 50% reduction in serum DPP-IV activity from baseline in untreated animals
³Not tested

### Example 6

This example illustrates that mammalian cells are relatively impermeable to Glu-boroPro compared to another potent dipeptidyl peptidase inhibitor, Val-boroPro.

### Materials and Methods

Intracellular Expression of Myc-Tagged Dipeptidyl Peptidase-8 (DPP-8) in 293T Cells

DPP-8 cDNA was amplified from cDNA prepared from RNA isolated by standard methods (as described in Example 1). The cDNA was prepared from 293T cells, but can be amplified from most cell types since DPP-8 is widely expressed (18). cDNA was cloned into a plasmid for preparation of 400 μg amounts for transfection experiments. Expression of the myc-tagged DPP-8 was achieved by transfection of the DPP-8-myc fusion plasmid into 293 T cells mediated by Lipofectamine 2000 transfection reagent as described in Example 1.

Post-Extraction Inhibition of DPP-8 by Amino Boronic Dipeptides

293 T cells transfected with myc-DPP-8 were extracted with 1% Triton-X and 150 μl of extract incubated at room temperature with either Glu-boroPro or Val-boroPro at a concentration of 5.3 μM or without additions. After 15 minutes, 0.6 μg of anti-myc monoclonal antibody (mAb 9E10, Becton-Dickinson) was added and the mixture incubated for 3 hours on ice. Each reaction mixture was then split into 3 aliquots of 48 μl and each aliquot mixed with of protein G coupled beads (Sigma Chemical Co., St. Louis, Mo.) in 600 μl Triton lysis buffer and incubated for 1 hour at 4° C. The beads were washed twice in Triton lysis buffer and twice in assay buffer (140 mM NaCl, 50 mM HEPES pH 8.1), warmed to room temperature, mixed with 500 μM Ala-Pro-AFC in assay buffer and incubated for 4 min. The enzymatic reactions were stopped by addition of 1 M sodium acetate and measured fluorometrically as described in Example 1.

Pre-Extraction Inhibition of Intracellular DPP-8 by Amino Boronic Dipeptides

Viable 293 T cells transfected with myc-DPP-8 plasmid approximately 48 hours previously were released by trypsin treatment, spun down and resuspended in the same medium (Freestyle 293 Expression medium (InVitrogen Corporation) containing 5% Fetal Calf serum (HyClone)). The cell suspension was incubated approximately 35 minutes in a non-tissue culture treated petri dish at 37° C./5% CO₂ to allow recovery before centrifugation and resuspension in the same medium at 5×10⁶ cells per ml. Aliquots (150 microlitre) were incubated with either Glu-boroPro or Val-boroPro at a concentration of 10⁻⁴ M or without additions for 30 minutes at 37° C. The cells were then chilled on ice, washed 3 times to remove the inhibitors, and extracted with 0.8 ml 1% Triton-X lysis buffer as above. Myc-DPP-8 was immunoprecipitated and dipeptidyl peptidase activity assayed fluorometrically with Ala-Pro-AFC substrate as described above for the post-extraction protocol; but instead of stopping the reactions with 1 M sodium acetate, fluorescence was monitored continuously in the fluorometer for 15 minutes after the addition of substrate.

Results:

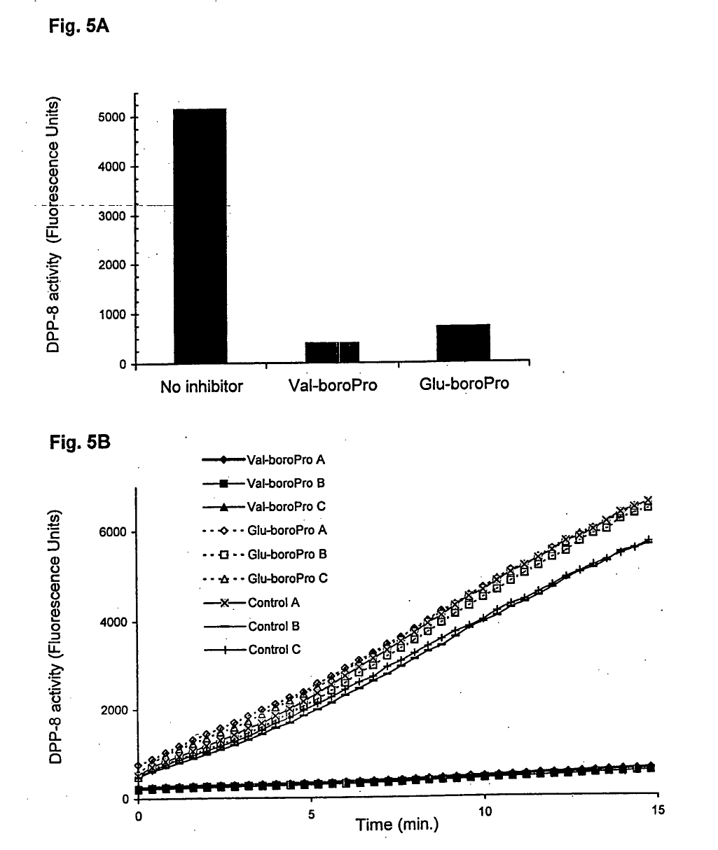

FIG. 5A illustrates the ability of 5.3-μM concentrations of both Val-boroPro and Glu-boroPro to inhibit the enzymatic activity of DPP-8 after extraction from myc-DPP-8 transfected 293 T cells. It should be noted that after incubation of cellular extracts with the amino boronic dipeptides, DPP-8 enzymatic activity remained inhibited after immunoprecipitation with anti-myc mAb. The relative stability of the complexes of DPP-8 and the amino boronic dipeptides demonstrated that intracellular DPP-8 could serve as an indicator of cell permeability to Val-boroPro and Glu-boroPro in the pre-extraction protocol. Utilizing this approach, in which intact, myc-DPP-8 transfected 293 T cells were incubated with the compounds before myc-DPP-8 was extracted, immunoprecipitated and assayed fluorometrically, it was found that 293 T cells were differentially permeable to Val-boroPro and Glu-boroPro. FIG. 5B illustrates that in triplicate samples (A, B and C) of myc-DPP-8 transfected 293 T cells incubated with 10⁻⁴ M concentrations of Val-boroPro or Glu-boroPro, only Val-boroPro appeared to enter the cells and inhibit intracellular DPP-8 activity.

### Example 7

This example illustrates that oral administration of Glu-boroPro to ob/ob mice 15 minutes prior to challenge by oral administration of glucose reduced the subsequent glucose excursion as indicated by determination of blood glucose levels.

### Materials and Methods

Animals

Male, 10-week old ob/ob mice (background: C57BLKS/J) were obtained from Charles River Laboratories (USA) and kept in a 12/12 hour light-dark cycle with controlled temperature conditions (22-24° C.). From time of arrival and throughout the experiment, mice were provided with standard rodent food (Altromin standard #1324 chow; C. Petersen, Ringsted, Denmark) and water ad libitum except were stated below.

Protocol for Mouse Oral Glucose Tolerance Test (OGTT)

The day of oral-glucose challenge was defined as day 0. On day −4, the mice were randomized (n=9 per group) to participate in one of the following drug-treatment groups: Group 1, vehicle (0.9% saline); Group 2, Glu-boroPro (1.0 μmol/kg). Agents were administered by oral gavage. Mice were restricted to a diet of 50% of their individual calculated food intake from day −1 onwards. On day 0, blood glucose was measured at t_{-15 min}. immediately followed by drug administration. At time point 0, glucose was administered by oral gavage (1 g/kg), and blood glucose was measured at time points 0, 30, 60, 120 and 240 minutes. Means±SE were calculated from the data of individual mice in drug-treatment groups 1 and 2. Statistical evaluation of the data was performed by one-way analysis of variance (ANOVA).

Results

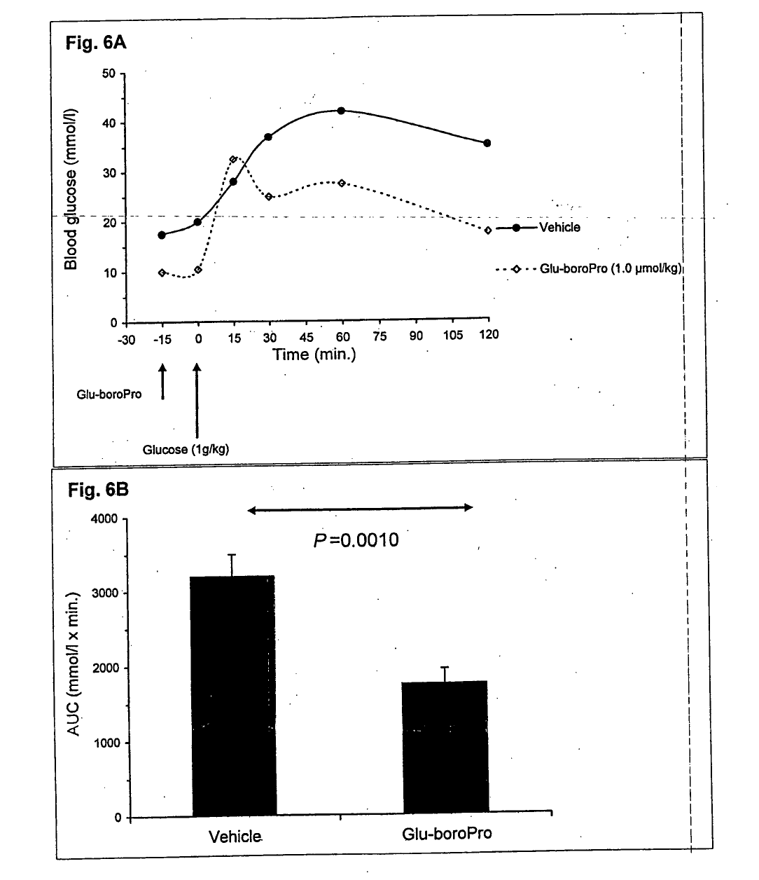

FIG. 6A illustrates the kinetic comparison of blood-glucose level between mice administered vehicle versus Glu-boroPro 15 minutes prior to oral glucose challenge. The glucose excursion post challenge was reduced by the Glu-boroPro treatment. Calculation of the area under the curves in FIG. 6B indicated that the anti-glycemic effect of the single 1.0 μmol/kg dose of Glu-boroPro was significant (P=0.0010).

### Example 8

This example illustrates that oral administration of Glu-boroPro to Zucker rats 15 minutes prior to challenge by oral administration of glucose reduced the subsequent glucose excursion, increased insulin and GLP-1 responses, and inhibited blood plasma DPP-IV activity, as indicated by the appropriate assays of blood levels.

### Materials and Methods

Animals

6-week old male Zucker ƒa/ƒa rats were obtained from Charles River Laboratories, USA) and housed in a 12/12 hour light-dark cycle with controlled temperature conditions (22-24° C.). From time of arrival and throughout the experiment, rats were provided with standard rodent food (Altromin standard #1324 chow; C. Petersen, Ringsted, Denmark) and water ad libitum except were stated below.

Protocol for Rat Oral Glucose Tolerance Test (OGTT)

The day of experimental oral-glucose challenge was defined as day 0. On days −11 to −8, rats were fitted with intra-arterial catheters under light isoflurane anesthesia. On day −1, the rats were stratified according to a randomization OGTT performed on day −6. Rats were randomized (n=6 per group) to participate in one of the following drug-treatment groups: Group 1, vehicle (0.9% saline); Group 2, Glu-boroPro (10.0 μmol/kg). From 12:00 a.m. (noon) on day −5, rats were offered only 50% of their individual 24-hour food intake. On day 0, drugs were administered by oral gavage at time point t_{-15 min} relative to time point 0 when glucose was administered by oral gavage (2 g/kg). Blood was sampled for analysis according to the following schedule, according to Table 3.

TABLE 3
Blood analysis scheme

| | | Blood sample volume (ml) collected for assay of: | | |
|---|---|---|---|---|
| | | Glucose and | | |
| Time point | | insulin | DPP-IV | GLP-1 |
| −15 | min. | 0.3 | | 0.5 |
| −5 | min. | 0.3 | 0.2 | |
| 0 | | 0.3 | 0.2 | 0.5 |
| 5 | min. | 0.3 | 0.2 | 0.5 |
| 10 | min. | 0.3 | | 0.5 |
| 15 | min. | 0.3 | | 0.5 |
| 20 | min. | 0.3 | 0.2 | |
| 30 | min. | 0.3 | | |
| 45 | min. | 0.3 | | |
| 60 | min. | 0.3 | | |
| 90 | min. | 0.3 | 0.2 | |
| 120 | min. | 0.3 | 0.2 | |
| 240 | min. | 0.3 | 0.2 | |
| 24 | hours | 0.3 | 0.2 | |
| 48 | hours | 0.3 | 0.2 | |

Serum DPP-IV activity was assayed fluorometrically as in Example 2, except that the substrate Gly-boroPro was substituted for Ala-boroPro as described elsewhere (11). Blood-plasma glucose was assayed with an automated analyzer (Roche Diagnostics). Active GLP-1 levels were determined in duplicate from each blood sample by ELISA (Linco Research, St. Charles, Mo.) and, similarly, P-insulin was measured by ELISA (Diamyd, Sweden). Means±SE were calculated from the data of individual rats in drug treatment groups 1 and 2. Statistical evaluation of the data was performed by one-way analysis of variance (ANOVA).

Results:

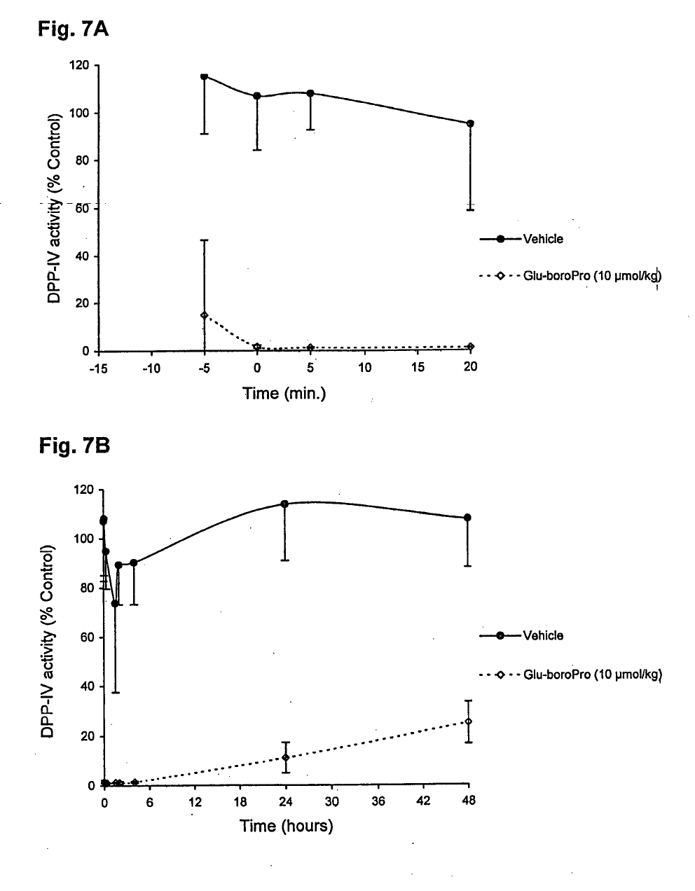

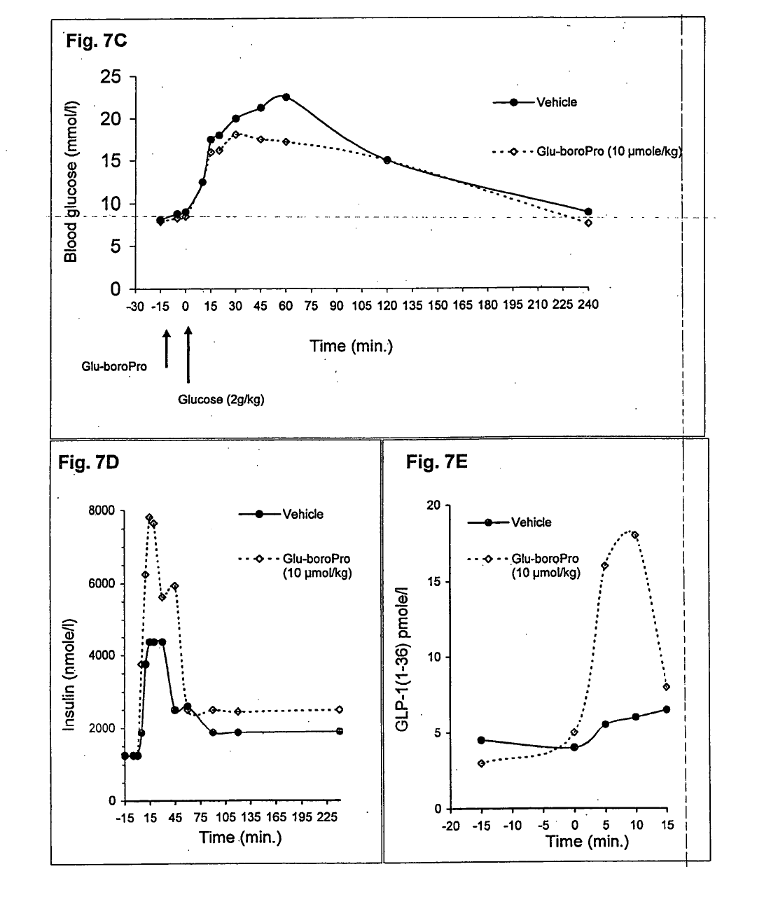

FIG. 7 illustrates the kinetics of serum DPP-IV inhibition following a single oral administration of a 10 μmol/kg dose of Glu-boroPro to Zucker rats at t₋₁₅ₘᵢₙ, relative to oral glucose challenge at time point 0. FIG. 7A illustrates DPP-IV activity at early time points: −5, 0, 5, and 20 minutes and FIG. 7B illustrates the complete kinetics up to the final measurement of DPP-IV activity at 48 hours. Marked inhibition of plasma DPP-IV activity was observed at t₋₅ₘᵢₙ and maximal inhibition was achieved by t₀ (FIG. 7A). Maximal inhibition of DPP-IV activity persisted until at least t₄ₕₒᵤᵣₛ (FIG. 7B). Plasma DPP-IV activity recovered to reach levels of 11% and 25% of control values at t₂₄ₕₒᵤᵣₛ and t₄₈ₕₒᵤᵣₛ, respectively.

FIG. 7C illustrates that blood glucose excursion was reduced by Glu-boroPro administration. DPP-IV inhibitors reduce blood glucose excursions in the OGTT by preventing the proteolytic degradation of GLP-1, which in turn results in an increased incretin effect on insulin secretion by pancreatic β-cells (4, 11-13, 19). In agreement with this mechanism of action, Glu-boroPro administration increased the blood plasma levels of both insulin and GLP-1 following oral glucose challenge in Zucker rats (FIGS. 7D and E). The inhibition of blood plasma DPP-IV activity observed after oral administration of the single dose of Glu-boroPro was clearly sufficiently rapid (FIG. 7A) to account for the increased levels of active GLP-1 (FIG. 7E).

### REFERENCES

1. Taylor, S. I. Deconstructing type 2 diabetes. Cell, 97: 9-12, 1999.
2. UKPDS UK prospective diabetes study 33: intensive blood glucose control with sulphonylureas or insulin compared with conventional treatment and risk of complications with type 2 diabetes. Lancet, 352: 837-853, 1998.
3. Drucker, D. J. The glucagon-like peptides. Endocrinology, 142: 521-527, 2001.
4. Moller, D. E. New drug targets for type 2 diabetes and metabolic syndrome. Nature, 414: 821-827, 2001.
5. de Meester, I., Lambeir, A. M., Proost, P., and Scharpe, S. Dipeptidyl peptidase IV substrates. An update on in vitro peptide hydrolysis by human DPP-IV. Adv. Exp. Med. Biol., 524: 3-17, 2003.
6. de Meester, I., Korom, S., Van Damme, J., and Scharpe, S. CD26, let it cut or cut it down. Immunol. Today, 20: 367-375, 1999.
7. Drucker, D. J. Biological actions and therapeutic potential of the glucagon-like peptides. Gastroenterology, 122: 531-544, 2002.
8. Marguet, D., Baggio, L., Kobayashi, T., Bernard, A.-M., Pierres, M., Nielsen, P. F., Ribel, U., Watanabe, T., Drucker, D. J., and Wagtmann, N. Enhanced insulin secretion an improved glucose tolerance in mice lacking CD26. Proc. Natl. Acad. Sci. USA, 97: 6874-6879, 2000.
9. Baikan, B., Kwasnik, L., Miserendino, R., Holst, J. J., and Li, X. Inhibition of dipeptidyl peptidase IV with NVP-DPP728 increases plasma GLP-1 (7-36 amide) concentrations and improves oral glucose tolerance in obese Zucker rats. Diabetologia, 42: 1324-1331, 1999.
10. Ahren, B., Holst, J. J., Martensson, H., and Balkan, B. Improved glucose tolerance and insulin secretion by inhibition of dipeptidyl peptidase IV in mice. Eur. J. Pharmacol., 404: 239-245, 2000.
11. Villhauer, E. B., Brinkman, J. A., Naderi, G. B., Burkey, B. F., Dunning, B. E., Prasad, K., Mangold, B. L., Russell, M. E., and Hughes, T. E. 1-[[3-Hydroxy-1-adamantyl)amino]acetyl]-2-cyano-(S)-pyrrolidine: a potent, selective, and orally bioavailable dipeptidyl peptidase IV inhibitor with antihyperglycemic properties. J. Med. Chem., 46: 2774-2789, 2003.
12. Ahren, B. and al, e. Inhibition of dipeptidyl peptidase IV improves metabolic control over a 4-week study period in type 2 diabetes. Diabetes Care, 25: 869-875, 2002.
13. Demuth, H.-U. e. a. Single dose treatment of diabetic patients by the DP-IV inhibitor P32/98. Diabetes, 49(Suppl. 1): A102, 2000.
14. Conarello, S. L., Li, Z., Ronan, J., Roy, R. S., Zhu, L., Jiang, G., Liu, F., Woods, J., Zycband, E., Moller, D. E., Thornberry, N. A., and Zhang, B. B. Mice lacking dipeptidyl peptidase IV are protected against obesity and insulin resistance. Proc. Natl. Acad. Sci. USA, 100: 6825-6830, 2003.
15. Durinx, C., Lambeir, A. M., E., B., Falmagne, J. B., Berghmans, R., A., H., Scharpe, S., and de Meester, I. Molecular characterization of dipeptidyl peptidase activity in serum: soluble CD26/dipeptidyl peptidase IV is responsible for the release of dipeptides. Eur. J. Biochem., 267: 5608-5613, 2000.
16. Jones, B., Adams, S., Miller, G. T., Jesson, M. I., Watanabe, T., and Wallner, B. P. Hematopoietic stimulation by a dipeptidyl peptidase inhibitor reveals a novel regulatory mechanism and therapeutic treatment for blood cell deficiencies. Blood, 102: 1641-1648, 2003.
17. Adams, S., Miller, G. T., Jesson, M. I., Watanabe, T., Jones, B., and Wallner, B. P. PT-100, a small molecule dipeptidyl peptidase inhibitor, has potent anti-tumor effects and augments antibody-mediated cytotoxicity via a novel immune mechanism. Cancer Res., 64: 5471-5480, 2004.
18. Abbott, C. A., Yu, D. M. T., Woollatt, E., Sutherland, G. R., and McCaughan, G. W. Cloning, expression and chromosomal localization of a novel human dipeptidyl peptidase (DPP) IV homolog, DPP8. Eur. J. Biochem., 267: 6140-6150, 2000.
19. Balkan, B., Kwasnik, L., Miserendino, R., Holst, J. J., and Li, X. Inhibition of dipeptidyl peptidase IV with NVP-DPP728 increases plasma GLP-1 (7-36 amide) concentrations and improves oral glucose tolerance in obese Zucker rats. Diabetologia, 42: 1324-1331, 1999.

The foregoing written specification is considered to be sufficient to enable one ordinarily skilled in the art to practice the invention. The present invention is not to be limited in scope by examples provided, since the examples are intended as mere illustrations of one or more aspects of the invention. Other functionally equivalent embodiments are considered within the scope of the invention. Various modifications of the invention in addition to those shown and described herein will become apparent to those skilled in the art from the foregoing description. Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth or illustrated in the drawings. The invention is capable of other embodiments and of being practiced or of being carried out in various ways.

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### Equivalents

It should be understood that the preceding is merely a detailed description of certain embodiments. It therefore should be apparent to those of ordinary skill in the art that various modifications and equivalents can be made without departing from the spirit and scope of the invention, and with no more than routine experimentation. It is intended to encompass all such modifications and equivalents within the scope of the appended claims.

All references, patents and patent applications that are recited in this application are incorporated by reference herein in their entirety.

What is claimed is:

1. A method for treating a subject having type 2 diabetes comprising
orally administering to a subject in need thereof, 15 minutes prior to glucose challenge, an agent having a structure of 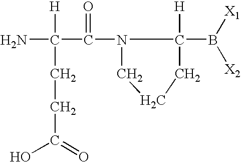
wherein each X₁ and X₂ is a hydroxyl group,
in an amount effective to reduce blood glucose level, after glucose challenge, by at least 40% relative to an untreated subject.
